(19) United States
(12) Patent Application Publication
(10) Pub. No.: US 2025/0304868 A1
(43) Pub. Date: Oct. 2, 2025

(54) OPTIMIZING FOSSIL AND SYNTHETIC RENEWABLE GASOLINE FUEL COMPOSITION FOR ULTRA-LEAN BURN ENGINES

(71) Applicants: King Abdullah University of Science and Technology, Thuwal-Jeddah (SA); Saudi Aramco Technologies Company, Dhahran (SA); Hyundai NGV, Seoul (KR)

(72) Inventors: Shashank Sakleshpur Nagaraja, Thuwal (SA); Balaji Mohan, Dhahran (SA); Junseok Chang, Dhahran (SA); S. Mani Sarathy, Thuwal (SA)

(73) Assignees: King Abdullah University of Science and Tech., Thuwal-Jeddah (SA); Saudi Aramco Technologies Company, Dhahran (SA)

(21) Appl. No.: 18/622,546

(22) Filed: Mar. 29, 2024

(51) Int. Cl. C10L 1/04; C10L 1/182; G16C 60/00

(52) U.S. Cl. CPC C10L 1/04; C10L 1/1824; G16C 60/00; C10L 2270/02; C10L 2290/60

(57) ABSTRACT

A composition that may be used as a fuel. The composition includes C₅-C₇ paraffins, in an amount not exceeding 20% by volume of the composition, C₅-C₉ iso-paraffins, in an amount from 30% to 90% by volume of the composition, C₅-C₈ olefins, in an amount not exceeding 40% by volume of the composition, C₅-C₁₀ naphthenes, in an amount not exceeding 20% by volume of the composition, C₅-C₁₀ aromatics, in an amount not exceeding 30% by volume of the composition, and a fuel additive comprising C₁-C₅ oxygenates, in an amount from 1% to 15% by volume of the composition.

### BACKGROUND

In modern mechanics, an increasing amount of effort has been spent on optimizing internal combustion engines.

Ultra-lean burn combustion engines, notably used in hybrid electric vehicles, have been developed to provide better efficiency and produce less NOₓ and CO₂ emissions compared to conventional combustion engines, while retaining similar performances.

To improve ultra-lean burn combustion engines further, it is desirable to design or optimize, possibly aided by artificial intelligence, fossil-based or synthetic renewable fuels in a way that will enhance these qualities.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

Embodiments disclosed herein generally relate to a composition that may be used as a fuel. The composition includes C₅-C₇ paraffins, in an amount not exceeding 20% by volume of the composition, C₅-C₉ iso-paraffins, in an amount from 30% to 90% by volume of the composition, C₅-C₈ olefins, in an amount not exceeding 40% by volume of the composition, C₅-C₁₀ naphthenes, in an amount not exceeding 20% by volume of the composition, C₅-C₁₀ aromatics, in an amount not exceeding 30% by volume of the composition, and a fuel additive comprising C₁-C₅ oxygenates, in an amount from 1% to 15% by volume of the composition.

Embodiments disclosed herein generally relate to a method for comparing and ranking materials according to their combustion. The method includes determining, using a computational model, values for one or more physical properties for each material within a plurality of materials, and determining a combustion score for each material, using a scoring function that receives as input the values of the one or more physical properties for the material. The method further includes creating an ordered list of the combustion scores sorted in decreasing order, each combustion score in the ordered list corresponding to a material and positioned at an index in the ordered list, where the index represents a suitability rank for the material to be used as a fuel in a combustion engine.

Embodiments disclosed herein generally relate to a method for optimizing concentrations of substances composing a material. The method includes obtaining a set of one or more physical properties influencing a combustion quality of a material in a combustion engine, obtaining a vector of N substances, where N is an integer greater than or equal to two, and obtaining a computational model configured to receive a material composed of the N substances, and output values for the one or more physical properties for the material, where each substance has a concentration within the material. The method further includes obtaining a scoring function configured to receive the values of the one or more physical properties for a material and output a combustion score for the material, and defining a Merit function that receives a vector of N concentrations as input and returns, as output, the combustion score of a material composed of the N substances, the substance at an index of the vector of N substances having the concentration at a same index from the vector of N concentrations, where the combustion score is the output of the scoring function that receives, as input, the one or more physical properties output by the computational model that receives the material as input. The method further includes computing, with an optimizer, an optimal vector of N concentrations, where the optimizer is configured to seek to maximize the Merit function.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1A depicts an example of a portion of a car, in accordance with one or more embodiments disclosed herein.

FIG. 1B depicts an example of a combustion engine, in accordance with one or more embodiments disclosed herein.

FIG. 2 depicts a system for assigning a score to a material, in accordance with one or more embodiments disclosed herein.

FIG. 3 depicts an example of a system for selecting a physical property, in accordance with one or more embodiments disclosed herein.

FIG. 4 depicts a schematic representation of an artificial intelligence model, in accordance with one or more embodiments disclosed herein.

FIG. 5 depicts a box diagram of system for determining minimum and maximum blending requirements of a fuel composition, in accordance with one or more embodiments disclosed herein.

FIG. 6 depicts an example of a box diagram of a system for producing a fuel, in accordance with one or more embodiments disclosed herein.

FIG. 7 depicts a flowchart of a method for ranking fuels, in accordance with one or more embodiments disclosed herein.

FIG. 8 depicts a flowchart of a method for optimizing concentrations of substances within a material, in accordance with one or more embodiments disclosed herein.

FIG. 9 depicts a flowchart of a method for training an AI model, in accordance with one or more embodiments disclosed herein.

FIG. 10 depicts a box diagram of a system for obtaining molecular representations, in accordance with one or more embodiments disclosed herein.

FIG. 11 depicts an example diagram of a neural network, in accordance with one or more embodiments disclosed herein.

FIG. 12 depicts an example diagram of a gradient boosted tree algorithm, in accordance with one or more embodiments disclosed herein.

FIG. 13 depicts an overview of training an artificial intelligence model, in accordance with one or more embodiments disclosed herein.

FIG. 14 depicts an example diagram of a computer, in accordance with one or more embodiments disclosed herein.

FIG. 15A depicts a scatter plot from an experiment, in accordance with one or more embodiments disclosed herein.

FIG. 15B depicts a scatter plot from an experiment, in accordance with one or more embodiments disclosed herein.

FIGS. 16A-16F depict results of testing an artificial intelligence model, in accordance with one or more embodiments disclosed herein.

FIG. 17 depicts a graph of combustion scores, in accordance with one or more embodiments disclosed herein.

FIG. 18A depicts a bar plot of an analysis of components of a fuel, in accordance with one or more embodiments disclosed herein.

FIG. 18B depicts a bar plot of an analysis of components of a fuel, in accordance with one or more embodiments disclosed herein.

FIGS. 19A-19C depict pie charts of an analysis of components of a fuel, in accordance with one or more embodiments disclosed herein.

FIGS. 20A-20C depict pie charts of an analysis of components of a fuel, in accordance with one or more embodiments disclosed herein.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. For example, a computer may reference two or more such computers.

As used here and in the appended claims, the words “comprise,” “has,” and “include” and all grammatical variations thereof are each intended to have an open, non-limiting meaning that does not exclude additional elements or steps.

“Optionally” means that the subsequently described event or circumstances may or may not occur. The description includes instances where the event or circumstance occurs and instances where it does not occur.

Terms such as “approximately,” “about,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. For example, these terms may mean that there can be a variance in value of up to ±10%, of up to 5%, of up to 2%, of up to 1%, of up to 0.5%, of up to 0.1%, or up to 0.01%.

Ranges may be expressed as from about one particular value to about another particular value, inclusive. When such a range is expressed, it is to be understood that another embodiment is from the one particular value to the other particular value, along with all particular values and combinations thereof within the range.

It is to be understood that one or more of the steps shown in a flowchart may be omitted, repeated, and/or performed in a different order than the order shown. Accordingly, the scope disclosed herein should not be considered limited to the specific arrangement of steps shown in the flowchart.

Although multiple dependent claims are not introduced, it would be apparent to one of ordinary skill that the subject matter of the dependent claims of one or more embodiments may be combined with other dependent claims.

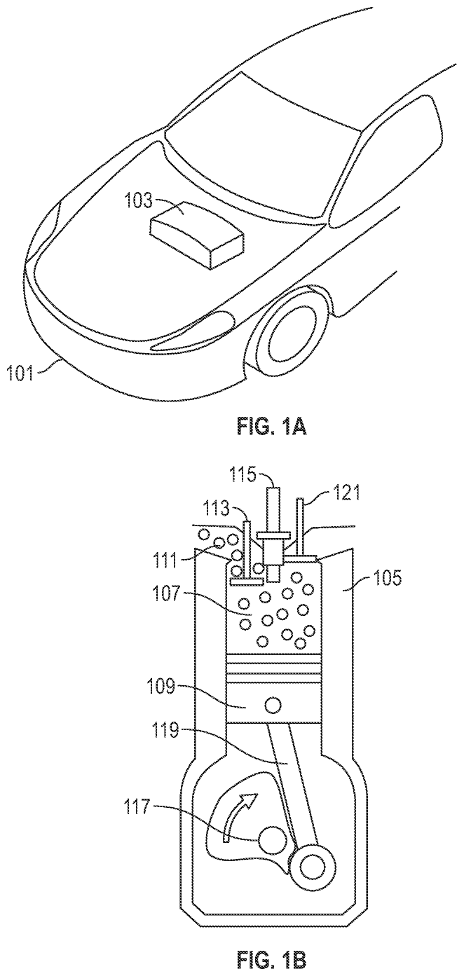

In the following description of FIGS. 1A-20C, any component described with regard to a figure, in various embodiments disclosed herein, may be equivalent to one or more like-named components described with regard to any other figure. For brevity, descriptions of these components will not be repeated with regard to each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described with regard to a corresponding like-named component in any other figure.

Hybrid electric vehicles (HEV) are vital for the global transition toward sustainable mobility, and ultra-lean burn (ULB) engines for HEVs are exemplary solutions due to their high efficiency, lower NOₓ emissions and reduced tank-to-wheel CO₂ emissions. To improve combustion efficiency and decrease emissions at ultra-lean conditions, it is crucial to identify appropriate properties and design compositions of fossil-based and/or synthetic renewable e-gasoline fuels that enhance these properties. Such properties are determined based on engine experiments. In one or more embodiments, based on these properties, a scoring function is formulated. Further, machine learning models are trained and utilized to predict the properties of more than 350,000 chemical species. Further, in embodiments disclosed herein, the scoring function is used to rank the chemical species, and the potential candidates are chosen using the high throughput screening approach.

FIG. 1A depicts a front portion of a car (101) that runs on a combustion engine (103), using fuel as a source of energy. Components of the combustion engine (103) may be formed of aluminum, iron, steel, or equivalent metals used in conventional engine design known to a person of ordinary skill in the art. The combustion engine (103) generally includes multiple cylinders, such as, for example, the cylinder (105) in FIG. 1B. In the embodiment in FIG. 1B, the cylinder (105) contains a combustion chamber (107) and a piston (109). A fuel-air mixture (111) is directed into the combustion chamber (107) through an intake valve (113). The fuel-air mixture (111) is ignited by a spark plug (115) and combusts in the combustion chamber (107), creating heat and producing exhaust gases, which expand and move the piston (109). The movement of the piston (109) may rotate a crankshaft (117), connected to the piston (109) by a rod (119). Thus, the chemical energy from the fuel-air mixture (111) is transformed into mechanical energy moving the crankshaft (117). The exhaust gases are released out of the cylinder (105) through an exhaust valve (121). The intake valve (113) and exhaust valve (121) may be actuated to open and closed positions according to an engine valve timing schedule. In situations in which the air-to-fuel ratio in the fuel-air mixture (111) is considered as high, the combustion may be qualified as an ultra-lean burn combustion, and the combustion engine (103) may then be called an ultra-lean burn combustion engine. In one or more embodiments, the air-to-fuel ratio in the fuel-air mixture (111) is considered as high if it is greater than a predefined ultra-lean burn threshold. A non-limiting example of an ultra-lean burn threshold above which the air-to-fuel ratio in the fuel-air mixture (111) may qualify the combustion as an ultra-lean burn combustion is 18:1. In one or more embodiments, an ultra-lean burn combustion is designed to improve fuel efficiency and reduce emissions of toxic or greenhouse gases, such as carbon dioxide (CO₂) or nitrogen oxides (NOₓ).

In one or more embodiments, a composition includes C₅-C₇ paraffins, in an amount not exceeding 20% by volume of the composition, C₅-C₉ iso-paraffins, in an amount from 30% to 90% by volume of the composition, C₅-C₈ olefins, in an amount not exceeding 40% by volume of the composition, C₅-C₁₀ naphthenes, in an amount not exceeding 20% by volume of the composition, C₅-C₁₀ aromatics, in an amount not exceeding 30% by volume of the composition, and a fuel additive comprising C₁-C₅ Oxygenates, in an amount from 1% to 15% by volume of the composition. In some embodiments of this composition, the olefins may be further classified into two categories and their respective amounts: C₅-C₇n-Olefins, in an amount not exceeding 20% by volume of the composition, and C₅-C₈ Iso-Olefins, in an amount not exceeding 20% by volume of the composition. In one or more embodiments, the fuel additive in this composition includes one or more alcohols. Examples of alcohols that may be included in the fuel additive include methanol, ethanol, iso-propanol, n-propanol, tert-butanol and any combinations thereof. In one or more embodiments, the fuel additive in this composition includes C₁-C₅ Oxygenates in respective amounts not more than as allowed by the regulatory standard EN228 as of the date of writing this disclosure, which imposes the mass concentration of oxygen, in a fuel composition, not to exceed 3.7%. As of the date of writing this disclosure, the regulatory standard EN228 further imposes the volume concentrations of methanol, in a fuel composition, not to exceed 3%, the ethanol volume concentration not to exceed 10%, the iso-propanol volume concentration not to exceed 12 v %, the iso-butanol volume concentration not to exceed 15%, the tert-butanol volume concentration not to exceed 15%, the ethers volume concentrations with five or more carbon atoms not to exceed 22%, and the volume concentration of other oxygenates not to exceed 15%. In one or more embodiments, the composition described in this paragraph may be used as a fuel for a combustion engine. A notable example of an engine in which this composition may be used as fuel is an ultra-lean burn combustion engine.

In this disclosure, the noun “material” is defined as a substance or a chemical composition of at least two substances. A combustible material may be used as fuel. However, the scope of this disclosure extends to any combustible material, even if it is not used as fuel. Furthermore, the combustion may occur anywhere such as, for example, in any type of engine, not necessarily in a car. Materials have physical properties, some of which can be measured of evaluated. Examples of physical properties for a material include a boiling point (BP), an adiabatic flame temperature (AFT), a laminar flame speed (LFS), a heat of vaporization (HOV), a carbon to oxygen ratio (C/O), a research octane number (RON), and a motor octane number (MON), that are defined in this disclosure in accordance with one or more embodiments. The BP of a material is a temperature at which the material changes from a liquid to a gas at a specific pressure. It varies depending on the pressure to which the material is exposed. For example, in some specific pressure conditions encountered on the surface of the Earth, the BP of ethanol is 78 degrees Celsius. The AFT of a material is a highest temperature that could be achieved during a combustion process if no heat was exchanged with the surroundings. For example, in some specific conditions including stochiometric proportions in presence with dioxygen (O₂), the AFT of methane is between 1949 and 1951 degrees Celsius. The LFS of a material is the speed at which a smooth, undisturbed flame front propagates through the material when combusted, that may be measured in a unit of distance over a unit of time. For example, in some specific conditions encountered at the surface of the Earth, the LFS of ethane is approximately in the range of 35 to 40 cm/s. The HOV of a material is an amount of heat energy required to transform a given quantity of the material from a liquid phase into a gaseous phase at a constant temperature and pressure, that may be expressed in a unit of energy such as joule, over a unit of mass. For example, in some specific conditions encountered at the surface of the Earth, the heat of vaporization of propane is 586000 J/kg. The C/O of a material is a ratio between a number of carbon atoms in the material and a number of oxygen atoms in the material. A notable example is the C/O of a material made of a single substance. For example, a molecule of ethanol is composed of two atoms of carbon, six atoms of hydrogen and one atom of oxygen, and therefore, the C/O of ethanol is two. The RON of a material is a measure of its resistance to detonating, under combustion conditions qualified as “mild”. The MON of a material is a measure of its resistance to detonating under combustion conditions qualified as “severe”. In one or more embodiments, combustion conditions are qualified as “mild” if a pressure in the environment where the combustion occurs is smaller than a pressure threshold, and as “severe” if a pressure in the environment where the combustion occurs is greater than the pressure threshold.

Generally, physical properties of the fuel from the fuel-air mixture (111) influence some properties of the combustion, termed as combustion properties. Examples of combustion properties include an efficiency, a performance and emissions of the combustion. The emissions of the combustion may be based on a quantity or a toxicity, or both, of the exhaust gases released from the combustion. Furthermore, throughout this disclosure, the general term “combustion quality” is used to define how suitable a material is to be combusted for a certain purpose. An example of purpose includes using the material as a fuel in a combustion engine. In that respect, fuels may be designed or optimized to improve the combustion quality. Efficiency, performance and emissions of a combustion may be defined in many ways. In some embodiments the efficiency, performance and emissions of a combustion are defined as in the following a), b) and c): a) efficiency is the inverse of a ratio between the chemical energy of the fuel that is burned in the combustion chamber (107) and the mechanical energy produced by the crankshaft (117), where an energy might be measured in joule; a notable example for the efficiency is an indicated thermal efficiency (ITE). b) the performance of a combustion is the amount of output power, that might be measured in Watt; c) the emission of the combustion is the volume of toxic or greenhouse gases released by the combustion, divided by the volume of fuel that is combusted. Examples of gases that may be released by a combustion include carbon monoxide (CO), carbon dioxide (CO₂), nitrogen oxides (NOₓ), such as nitric oxide (NO) and nitrogen dioxide (NO₂), partially burned hydrocarbons from the fuel molecules, and sulfur dioxide (SO₂). It is emphasized that the examples of definitions of efficiency, performance and emissions of a combustion are given in this paragraph only as examples and should not be considered limiting. One with ordinary skill in the art will recognize that other examples of definitions of efficiency, performance and emissions of a combustion may be used without departing from the scope of this disclosure.

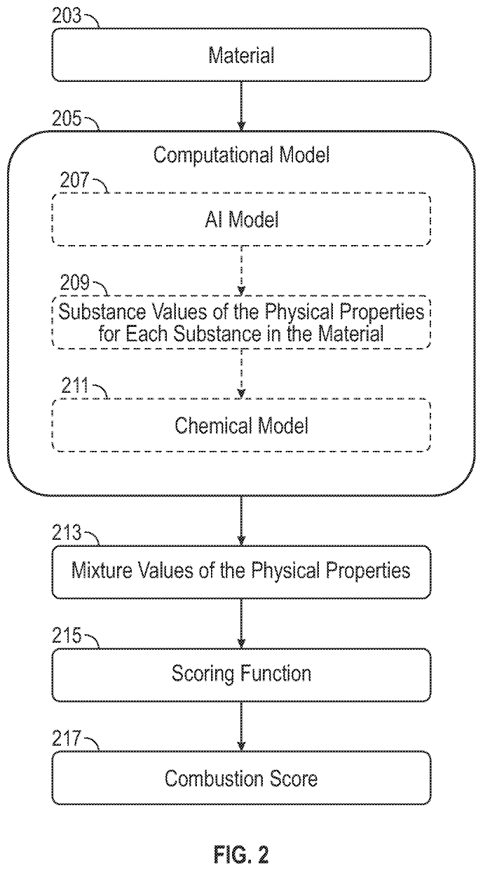

FIG. 2 depicts a system to obtain a combustion score of a material (203). The material (203), comprising one or more substances, and the concentrations of each substance, are passed as input to a computational model (205). In one or more embodiments, the input to the computational model is a set of representations of all molecules that make up the material (203), and the set of the concentrations of each substance. Examples of representations of a molecule include an empirical formula, a structural formula, a Lewis structure and a Simplified Molecular Input Line Entry System (SMILES) representation. An empirical formula of a molecule lists the atoms present in the molecule, as well as the number of each atom present. For example, the empirical formula of methane, composed of one atom of carbon and four atoms of hydrogen is CH₄. A structural formula of a molecule may be defined as a graphic representation of the molecular structure, showing how the atoms composing the molecule are arranged in three space dimensions. A structural formula may include bonds between atoms of the molecule, angles formed between imaginary lines connecting the atoms of the molecule, electrical charges of the atoms, and stereochemistry indicators, such indicators being drawn as a wedge. A Lewis structure of a molecule is a representation of a molecule that uses dots to represent the valence electrons of atoms and lines to represent chemical bonds, hence providing information about the connectivity of atoms in the molecule, without disclosing any three-dimensional information. For example, the Lewis structure for carbon monoxide is :C≡O:. A SMILES representation of a molecule is a line notation that represents the atoms and structure of the molecule using ASCII characters. Although describing all aspects of SMILES extends beyond the scope of this disclosure, a few components of SMILES are described herein. Each atom is represented by its symbol from a periodic table of the elements, the symbol being written between brackets. For example, the SMILES representation for gold is [Au]. Brackets may be omitted for some elements, such as carbon, oxygen and nitrogen. For example, a SMILES representation for carbon is C, and another SMILES representation for carbon is [C]. In some instances, the hydrogen element may be omitted in the SMILES representation if the hydrogen element is connected with a single bond to other, specific elements such as oxygen and carbon. Thus, examples of SMILES representations for water include O, [OH2] and [H]O[H]. In some SMILES representations, a single bond is either represented as the symbol “-” or omitted. In that regard, SMILES representations for ethanol include C—C—O, CC—O and CCO.

The computational model (205) returns values for physical properties, within a set of one or more physical properties, for the material (203) as outputs, also called mixture values of the physical properties (213). In one or more embodiments, the computational model includes a physical model that connects material (203) as an input to an output value of a physical property by using laws of physics. Such a physical model may be of various forms, including a formula that provides a value of the output directly, or an equation that needs to be solved to find the output, such as a numerical equation, a differential equation or an integral equation. In that respect, the computational model (205) may further include methods to solve an equation, such as an iterative solver or a numerical method. Examples of iterative solvers include Newton methods and pseudo-Newton methods, which seek the solution of a non-linear equation by computing a sequence that is intended to converge towards a solution to an equation. Numerical methods include quadrature formulas that approximate integrals, such as a method of rectangles or Simpson's rule. Numerical methods further include discretization methods for differential equations, such as Runge-Kutta methods, finite differences and finite element methods. In this disclosure, a physical model may also include, or be called, a mathematical model.

In one or more embodiments, the computational model (205) makes use of artificial intelligence (AI) in the form of an AI model (207). Examples of an AI model (207) that predict the mixture values of the physical properties (213) from the material (203) include supervised machine learning models of various types. If a physical property to be predicted is a numerical value, such as boiling point or adiabatic flame temperature, the physical property may be referred to as a “numerical physical property”, and examples of supervised machine learning models that may be used in the computational model include regression models, such as a linear regression or a polynomial regression. If a physical property to be predicted is a category, rather than a numerical quantity, the physical property may be referred to as “categorical physical property”, and examples of supervised machine learning models that may be used in the computational model include classification models, such as logistic regression models, decision trees and support vector machines.

It is noted that depending on the purpose of determining the mixture values of the physical properties (213) for the material (203), a numerical physical property may be transformed into a categorical physical property by means of binning. Binning a set of numbers that takes values within a certain interval includes splitting the interval into sub-intervals, giving each sub-interval a name, finding to which interval each number from the set of numbers belongs, and for each number within the set of numbers, assigning a category to the number, the category being the name of the sub-interval to which the number belongs. An example of three interval binning for the boiling point of a substance is given by defining two boiling thresholds, namely BT1 and BT2, such that BT1<BT2. Then, the boiling point of a substance may be qualified as “low” if it is less than BT1, “medium” if it is greater than or equal to BT1 and less than BT2, or “high” if it is greater than or equal to BT2. Such a resulting binned boiling point, of “low”, “medium” or “high”, is a categorical physical property. It is noted that the AI models given herein are intended to provide some examples only. Several other AI models may be used without departing from the scope of this disclosure.

Further examples of an AI model (207) that may be included in the computational model (205) include neural networks (NN), such as deep neural networks (DNN), convolutional neural networks (CNN) or recurrent neural networks (RNN). In one or more embodiments, a supervised machine learning model included in the computational model is trained by using training examples, where each training example is an input-output pair, in which an input is a representation of a material2, and the output is a set of mixture values of the physical properties that are already known for the material2. In one or more embodiments, mixture values of the physical properties, for a training example, may be determined, for a material2 by doing experiments on the material (203) and measuring the values of the physical properties with measuring instruments, such as sensors. After the supervised machine learning model has been trained, it may receive a representation of the material (203) as input and predict, as output, the mixture values of the physical properties (213) by means of computing, rather than running experiments.

The AI model (207) may be trained to compute all of the mixture values of the physical properties (213) together, of the material (203), or it may be trained to compute a value for one physical property at a time. In one or more embodiments, the AI model (207) is trained to compute values for the physical properties of each substance composing the material, each substance made of one molecule. Then, in the general case where the material (203) is composed of more than one substance, the AI model (207) computes values of the physical properties for each substance composing the material (203) referred to as substance values of the physical properties (209). Then, the substance values of the physical properties (209) are combined into the mixture values of the physical properties (213) by using a chemical model (211), that receives the substance values of the physical properties (209) and the concentrations of each substance within the material (203) as inputs. An example for the chemical model (211) is a linear mole fraction model. Denoting Pᵢ, i=1, . . . , N as the value of a physical property for each of N substances Sᵢ, for i=1, . . . , N, composing a material, where N is a positive integer, and denoting Cᵢ, i=1, . . . , N as the concentration of the substance Sᵢ within the material, for i=1, . . . , N such that Σ₁^{N} Cᵢ=1, the value of the physical property for the material is given by the linear mole fraction approach as

$$P = {\sum }_{1}^{N} {C}_{i} {P}_{i} . \qquad EQ . 1$$

In one or more embodiments, the chemical model (211) makes also use of AI. Note that if the specific situation where the material (203) is composed of a single substance, the chemical model (211) is either omitted or is reduced to the identity, and the mixture values of the physical properties (213) are equal to the substance values of the physical properties (209).

The mixture values of the physical properties (213) are passed on to a scoring function (215) that returns, as output, a combustion score (217) for the material (203). In one or more embodiments, the scoring function (215) includes one or more factors selected from the group consisting of an efficiency factor, an emissions factor, and a performance factor. A notable example of such a scoring function (215) is a convex combination, L(P), of an efficiency factor A(P), an emissions factor B(P), and a performance factor C(P), that each receive a set of mixture values of physical properties denoted as P, the scoring function thus defined as:

$$L ( P ) = {w}_{1} A ( P ) + {w}_{2} B ( P ) + {w}_{3} C ( P ) , \qquad EQ . 2$$

where w₁, w₂ and w₃ are three nonnegative real numbers such that w₁+w₂+w₃=1. Note that EQ. 2 is to be understood as

$$“ L ( P ) = {w}_{1} \cdot efficiency ( P ) + {w}_{2} \cdot emissions ( P ) + {w}_{3} \cdot performance ( P ) ” .$$

It will be understood that as multivariable functions, the terms A(P), B(P) and C(P) in EQ. 2 need not depend on all the physical properties included in P. Further, it will be understood that other factors representing other combustion properties may be used in determining the scoring function. For example, in EQ. 2 the efficiency factor, the emissions factor, and the performance factor are exemplary of a first, second, and third combustion property factor, each of which has an associated weighting factor in contributing to the combustion score. It will be understood that combustion properties may be experimentally measurable and may depend on one or more of the physical properties. Thus, a selection of physical properties to use may depend on the choice of combustion properties accounted for in the factors.

As used herein, L is illustrative of the scoring function and L(P) is illustrative of the combustion score. In EQ. 2, the coefficients w₁, w₂ and w₃ may be pre-defined according to the purpose of scoring the material (203). For example, to give equal importance to each of the efficiency, emissions and performance factors, the coefficients w₁, w₂ and w₃ can be set to

$${w}_{1} = {w}_{2} = {w}_{3} = \frac{1}{3} .$$

In other scenarios, an emphasis can be put on some of the efficiency, emissions or performance factors by setting some the coefficients w₁, w₂ and w₃ small for the other factors. For example, by setting w₁=0.8 and w₂=w₃=0.1, the combustion score (217) has a stronger dependency on the efficiency factor than the emissions or performance factors. In an extreme case, some of the coefficients w₁, w₂ and w₃ can be set to 0 so that some of the efficiency, emissions and performance factors are neglected. For example, by setting w₂=1 and w₁=w₃=0, the combustion score (217) only takes emissions into account. Taking only emissions into account in the scoring function (215) may be useful in case fuels are to be ranked only according to how much gases are emitted during their combustion.

In one or more embodiments, the efficiency, emissions and a performance factors A, B and C are defined so that the combustion score (217) increases with an increase of the efficiency factor A(P), emissions factor B(P), or performance factor C(P). It is noted that the emissions factor B(P) is not necessarily intended to be seen as an amount of gas emitted by the combustion of the material (203), but rather a number that evaluates the amount of gas emitted by the combustion of the material (203). As such, the emissions factor B(P) may be designed to increase for a decrease in the toxic or greenhouse gas emissions from the combustion of the material (203). It is noted that there are many ways of defining the scoring function (215). The example of the scoring function (215) given in EQ. 2, as a convex combination of an efficiency factor, an emissions factor and a performance factor, is given as an example only and should not be considered limiting. Many other ways of defining the scoring function (215) may be used without departing the scope of this disclosure. In some embodiments, the scoring function (215) may be a non-linear combination of the efficiency, emissions and performance factors. In other embodiments, the scoring function (215) may include other factors, not related to efficiency, emissions or performance. In further embodiments, the scoring function (215) may not be separable into factors representing any combustion quantities

Examples of efficiency factor A, emissions factor B, and performance factor C that may be used in EQ. 2 include:

$$A ( P ) = \frac{170.3 - 0.95 * BP}{190} + \{ \begin{matrix}1 if L F S > 21 cm \text{/s} \\ 0 otherwise\end{matrix} , \qquad EQ . 3$$

$$B ( P ) = \frac{1630 - A F T}{5} + \frac{3.45 - log ( C / O )}{0.6} + \frac{H O V - 487}{50} , \qquad EQ . 4$$

$$C ( P ) = \frac{RON - 0.3 ( RON - MON ) - 92.24}{6} . \qquad EQ . 5$$

In EQ. 3, EQ. 4 and EQ. 5, BP is the boiling point of the material in degree Celsius, LFS is the laminar flame speed of the material with an air-fuel ratio of 1.8 and an initial temperature of 358K, AFT is the adiabatic flame temperature, in degree Kelvin, of the material, with an air-fuel ratio of 1.8, C/O is the carbon-oxygen ratio of the material, HOV is heat of vaporization of the material in KJ/kg, RON is the research octane number of the material, MON is the motor octane number of the material, and the set P of physical properties is defined as P=(BP,LFS,AFT,C/O,HOV,RON,MON). In the example given by EQ. 3, EQ. 4 and EQ. 5, the set P of physical properties is defined as P=(BP,LFS,AFT,C/O,HOV,RON,MON). However, in EQ. 3, EQ. 4 and EQ. 5, the terms A(P), B(P) and C(P) do not depend on all the physical properties included in P. The term A(P) only depends on BP and LFS, the term B(P) only depends on AFT, C/O and HOV and the term C(P) only depends on RON and MON. The scoring function (215) is not assumed to be dependent on any other physical properties in the specific embodiment in EQ. 3, EQ. 4 and EQ. 5. The formulas defining the efficiency, emissions and performance factors in EQ. 3, EQ. 4 and EQ. 5 are given as examples only and should not be considered as limiting the scope of this disclosure. Many other formulas for defining the efficiency, emissions and performance factors may be used without departing from the scope of this disclosure.

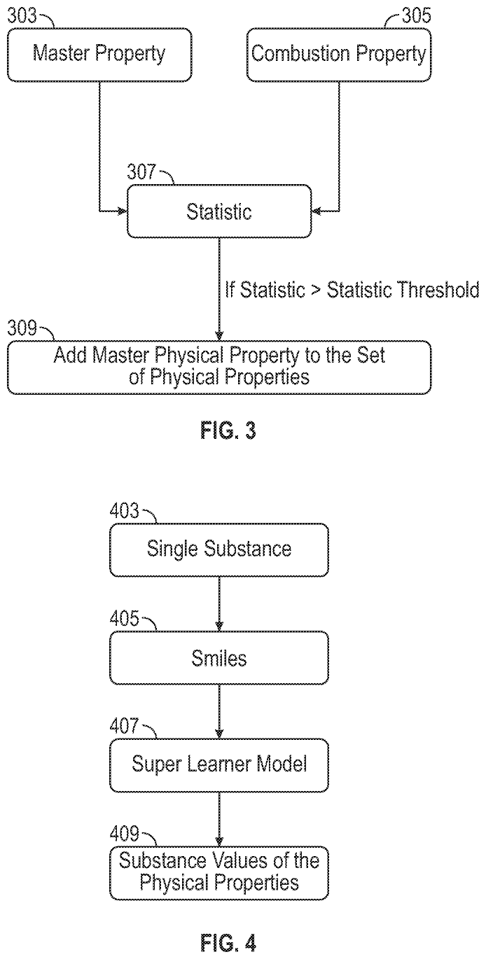

FIG. 3 depicts a system for defining the physical properties that are used in this disclosure, according to one or more embodiments. In this method, the physical properties are selected from a predefined set of properties, referred to as “master properties.” The master properties are pre-defined candidates to be selected as physical properties if they correlate with the combustion of the materials. Examples of master properties include a boiling point, a heat of formation, a solubility, a laminar flame speed, a research octane number, a cetane number, and a yield sooting index. In the system in FIG. 3, a broad set of master properties is analyzed through a set of experiments, and relevant physical properties are picked from the set of master properties only if they correlate with the combustion of the material. Each experiment consists of burning, in a combustion engine, several materials with a varying master property, measuring the value of a combustion property obtained through the combustion, and determining whether a correlation exists between the master property and the combustion property. If a correlation exists between the master property and the combustion property, the master property is included in the set of physical properties. If no correlation exists between the master property and the combustion property, the master property is not selected as a physical property.

Initially in FIG. 3, there is no certainty whether any master property correlates with any combustion property and therefore, at first, a set of physical properties is initialized as an empty set. A master property (303) is selected randomly from the set of master properties. A combustion property (305) is further selected to be measured in each experiment. Examples of a combustion property (305) include an efficiency of the combustion, such as an indicated thermal efficiency (ITE). Examples of a combustion property (305) further include a performance or an emission of the combustion, such as a NOₓ emission. To decide whether the master property (303) should be added to the set of physical properties, values of the master property (303) are determined for a set of N materials, Fᵢ, i=1, . . . N, with N≥2, resulting in N values for the master property (303). For example, if the master property (303) is a boiling point value of the master property (303) for a material may be determined by heating the material until it boils, and record the temperature at which it boils. Then, the N materials are combusted separately, and a value of the combustion property (305) is measured for each of the N combustions, resulting in N values for the combustion property (305). In some implementations, the combustion property (305) is measured using a measuring equipment, such as sensors or a chromatography instrument. For example, if the combustion property (305) is a NOₓ emission, a possible method to measure the NOₓ emission from the combustion of a material is to collect the exhaust gases from the combustion and determine the amount of NOₓ within the collected gases by using chromatography. Following the experiment, a statistic (307) is computed between the master property (303) and the combustion property (305), using the N values for the master property (303) and the N values for the combustion property (305).

Two examples for the statistic (307) are described herein. In one or more embodiments, the statistic (307) between the master property (303) and the combustion property (305) is computed independently from any other master properties of the material. In this case, denoting yᵢ as the value of the master property (303) for the i^{th} material Fᵢ, for i=1, . . . N and yᵢ as the value of the combustion property obtained by combusting Fᵢ, the statistic (307) is computed as a function of the pairs (xᵢ, yᵢ). Examples of the statistic (307) computed as a function of the pairs (xᵢ, yᵢ) include a Pearson's correlation coefficient:

$$r = \frac{{\sum }_{i = 1}^{i = N} ( {x}_{i} - \overline{x} ) ( {y}_{i} - \overline{y} )}{\sqrt{{\sum }_{i = 1}^{i = N} {( {x}_{i} - \overline{x} )}^{2}} \sqrt{{\sum }_{i = 1}^{i = N} {( {y}_{i} - \overline{y} )}^{2}}} . \qquad EQ . 6$$

### In EQ. 6,

$$\overline{x} = \frac{1}{N} {\sum }_{i = 1}^{i = N} {x}_{i} and \overline{y} = \frac{1}{N} {\sum }_{i = 1}^{i = N} {y}_{i} .$$

It is noted that this example of computing the statistic (307) assumes that the combustion property (305) only depends on the master property (303), and does not depend on any other master properties of the material. Thus, in some embodiments, using EQ. 6 as the statistic (307) is considered relevant if the N materials are properly selected so that any master property, aside from the master property (303), has similar values for the N materials. Denoting zᵢ as a value of a master property for the material Fᵢ, for i=1, . . . N, the master property is said to have similar values for the N materials if a value of a similarity between the values of the zᵢ, for i=1, . . . N, is less than a predefined threshold, for a predefined similarity metric. An example of a similarity metric between the values zᵢ, for i=1, . . . N, that may be used to compute the similarity between the values of the zᵢ, for i=1, . . . N, is a mean absolute distance

$$\frac{1}{N} \frac{1}{{z}_{max}} {\sum }_{i = 1}^{i = N} {\sum }_{j = 1}^{j = N} \vert {z}_{i} - {z}_{j} \vert ,$$

where zₘₐₓ=max_{i=1, . . . , N}(zᵢ).

In other embodiments, the statistic (307), between the master property (303) and the combustion property (305), is computed by taking into account values of other master properties of the material. An example of such statistic (307) is a multiple Pearson correlation. In this scenario, the master properties in the set of master properties are denoted as Mⱼ, j=1, . . . M, where M is the number of master properties in the set of master properties. Values are obtained for each of the M master properties, for each of the N materials. Denoting xᵢ^{j} as the value of the master property Mi for the material Fᵢ, for i=1, . . . N, j=1, . . . M,a correlation matrix R is defined by its M×M elements as

$${r}_{jk} = \frac{{\sum }_{i = 1}^{i = N} ( {x}_{i}^{j} - {\overline{x}}^{j} ) ( {x}_{i}^{k} - {\overline{x}}^{k} )}{\sqrt{{\sum }_{i = 1}^{i = N} {( {x}_{i}^{j} - {\overline{x}}^{j} )}^{2}} \sqrt{{\sum }_{i = 1}^{i = N} {( {x}_{i}^{k} - {\overline{x}}^{k} )}^{2}}} , \qquad EQ . 7$$

for j=1, . . . M, k=1, . . . M. In EQ. 7,

$${\overline{x}}^{j} = \frac{1}{N} {\sum }_{i = 1}^{i = N} {x}_{i}^{j} ,$$

for j=1, . . . M. Then, denoting V as the M-dimensional vector with components

$${r}_{j} = \frac{{\sum }_{i = 1}^{i = N} ( {x}_{i}^{j} - {\overline{x}}^{j} ) ( {y}_{i} - \overline{y} )}{\sqrt{{\sum }_{i = 1}^{i = N} {( {x}_{i}^{j} - {\overline{x}}^{j} )}^{2}} \sqrt{{\sum }_{i = 1}^{i = N} {( {y}_{i} - \overline{y} )}^{2}}} , \qquad EQ . 8$$

for j=1, . . . M, the multiple Pearson correlation vector is defined as E=V^{T}R⁻¹V, where R⁻¹ is the inverse of the matrix R. In EQ. 8, yᵢ is the value of the combustion property (305) obtained by combusting the material Fᵢ. Denoting/as an index such that the master property (303) is the master property M_{J}, the multiple correlation value, that may be used as the statistic (307), between the master property (303) and the combustion property (305), is defined as the square root of the J^{th} component, E_{J}, of the vector E.

Continuing with FIG. 3, the statistic (307) between the master property (303) and the combustion property (305) is compared to a certain, pre-defined statistic threshold. In one or more embodiments, if the computed statistic (307) is greater than the statistic threshold, the master property (303) is added, through an inclusion action (309), to the set of physical properties. The set of physical properties is completely determined by repeating the system in FIG. 3 for all the master properties within the set of master properties.

FIG. 4 depicts a high-level overview of an embodiment of the AI model (207) from FIG. 2. In this specific embodiment, the AI model (207) is a super learner model (407) that receives a SMILES representation (405) of a single molecule, representing a single substance (403), as input, and returns substance values of the physical properties (409) of the single substance (403) as output. The super learner model (407) is built as an ensemble learner model combining a set of multiple machine learning models called base learner models. Examples of super learner models include bagging algorithms, boosting algorithms, and voting algorithms. In bagging algorithms, multiple instances of the same base learner model are trained on different subsets of the training data. The final prediction may be defined as an average or a maximum voting score of the predictions from each instance. In boosting algorithms, base learner models are run sequentially, each base learner in the sequence correcting for errors made by the previous base learners in the sequence. Examples of boosting algorithms include adaboost, gradient boosting algorithms and catboost. Voting algorithms include running the base learner models separately using the whole training dataset and defining the output from the super learner model (407) as the output that was obtained the most times from the different base learner models. In one or more embodiments, the base learner models are selected from a pool of 40 machine learning models, that may include decision tree regressors, random forest regressors, support vector machine models and regression models, and base learners of different types may be included in the super learner model (407). In one or more embodiments, the training of the super learner model is performed by using a Sequential Least-Squares Programming method.

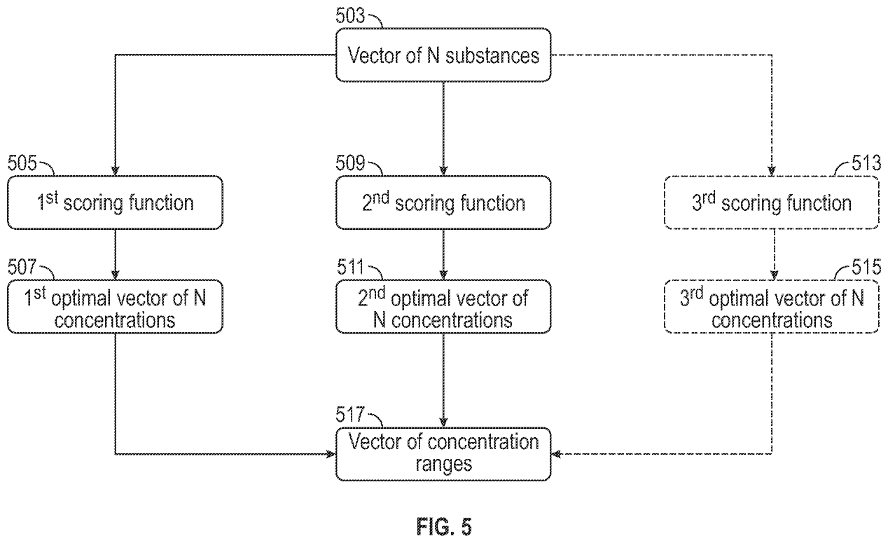

FIG. 5 depicts a system for determining ranges for concentrations of different substances composing a material, the ranges being obtained as optimum for a collection of scoring functions, as defined herein. A first optimal vector of N concentrations {C_{1,i}, i=1, . . . , N} (507) is computed for a vector of N substances (503) and a first scoring function (505) is obtained. A second optimal vector of N concentrations (511), {C_{2,i}, i=1, . . . , N}, is obtained and a second scoring function (509) is obtained, the second scoring function (509) being different from the first scoring function (505). A vector of N concentration ranges (517) is created, that defines minimum and maximum blending requirements to form a material from the N substances from the vector of N substances (503). The minimum and maximum blending requirements are based on the first optimal vector of N concentrations (507), {C_{1,i}, i=1, . . . , N}, and the second optimal vector of N concentrations (511), {C_{2,i}, i=1, . . . , N}. The vector of concentration ranges (517) is defined as the N-dimensional vector R such that each component Rᵢ of the vector R is the range [min(C_{1,i}, C_{2,i}), max(C_{1,i}, C_{2,i})], for i=1, . . . , N. This method extends to obtaining minimum and maximum blending requirements for the N substances, using more than two different scoring functions. A third optimal vector of N concentrations (515), {C_{3,i}, i=1, . . . , N}, is computed, and minimum and maximum blending requirements to form a material from the N substances from the vector of N substances (503) are determined as the N-dimensional vector of concentration ranges R (517) such that each component Rᵢ of the vector R is the range [min(C_{1,i}, C_{2,i}, C_{3,i}), max(C_{1,i}, C_{2,i}, C_{3,i})], for i=1, . . . , N. Extending the method further, assume that an integer number K of optimal vectors of N concentrations, {C_{k,i}, i=1, . . . , N} are obtained, for k=1, . . . , K. Minimum and maximum blending requirements to form a material from the N substances from the vector of N substances (503) are determined as the N-dimensional vector of concentration ranges R (517) such that each component Rᵢ of the vector R is the range [min(C₁, C₂, . . . , C_{K,i}), max(C_{1,i}, C_{2,i}, . . . , C_{k,i})], for i=1, . . . , N. It will be understood that while three scoring functions are shown in FIG. 5, a system for determining ranges for concentrations of different substances composing a material may use an alternate number of scoring functions that is two or greater.

In one or more embodiments, a master scoring function is defined by a formula that includes parameters. Then, the first scoring function (505), the second scoring function (509), and, by extension, the third scoring function (513), and any other scoring function used in the description of FIG. 5 are obtained by varying the parameters in the formula. In one or more embodiments, the master scoring function is defined by the formula in EQ. 2, that include the parameters w₁, w₂ and w₃, and the first scoring function (505), the second scoring function (509), and, by extension, the third scoring function (513), and any other scoring function used in the description of FIG. 5 are obtained by varying the parameters w₁, w₂ and w₃. In one or more embodiments, the efficiency factor A(P), emissions factor B(P), and performance factor C(P) in the master formula given by EQ. 2 are given by EQ. 3, EQ. 4 and EQ. 5.

In one or more embodiments, the system in FIG. 5 leads to the blending concentration ranges for a composition previously defined in this disclosure, including C₅-C₇ paraffins, in an amount not exceeding 20% by volume of the composition, C₅-C₉ iso-paraffins, in an amount from 30% to 90% by volume of the composition, C₅-C₈ olefins, in an amount not exceeding 40% by volume of the composition, C₅-C₁₀ naphthenes, in an amount from 1% to 20% by volume of the composition, C₅-C₁₀ aromatics, in an amount not exceeding 30% by volume of the composition, and a fuel additive comprising C₁-C₅ Oxygenates, in an amount from 1% to 15% by volume of the composition.

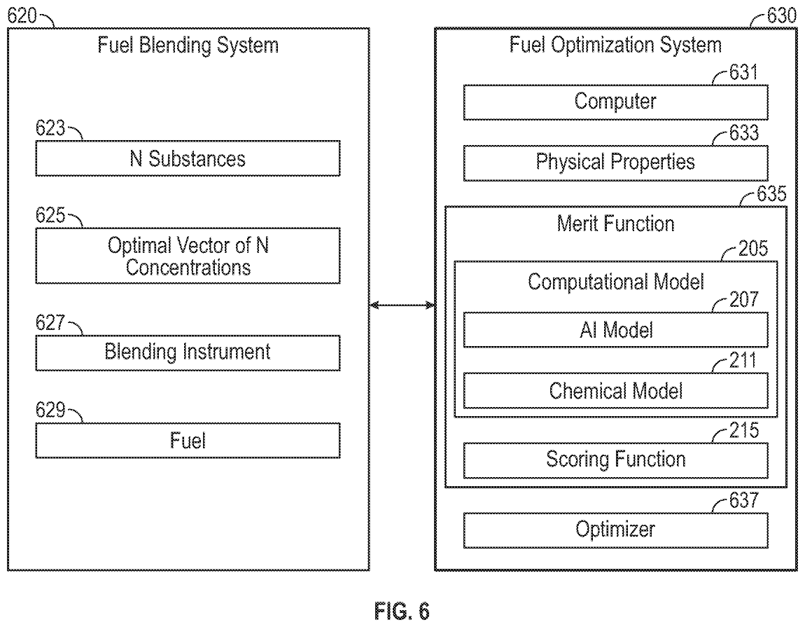

FIG. 6 depicts a system to produce a fuel that may be used in a combustion engine. For concision, a full description of components and/or elements depicted in FIG. 6 is not provided anew for those components and/elements that have be previously described with reference to the preceding figures. FIG. 6 includes a fuel blending system (620) and a fuel optimization system (630). The fuel blending system (620) receives N substances (623), denoted by Sᵢ, i=1, . . . , N, that are passed on to the fuel optimization system (630). The fuel optimization system (630) includes a list of physical properties (633) and a Merit function (635), that, as defined in EQ. 16, is composed of the scoring function (215), applied to the output of the computational model (205). The computational model (205) is itself composed of the AI model (207) and the chemical model (211). The AI model (207) is used to compute values of the physical properties in the list of physical properties (633) for each substance within the N substances (623). Given a vector of N concentrations Cᵢ, i=1, . . . , N such that Σ₁^{N} Cᵢ=1 and the values of the physical properties in the list of physical properties (633) obtained from the AI model (207), the chemical model (211) is configured to output the values of the physical properties in the list of physical properties (633) for the material {(Sᵢ, Cᵢ), i=1, . . . , N}.

In one or more embodiments, the Merit function (635) is formed by applying the scoring function (215) to the values of the physical properties for the material {(Sᵢ, Cᵢ), i=1, . . . , N}. As such, the Merit function receives a vector of N concentrations Cᵢ, i=1, . . . , N such that Σ₁^{N} Cᵢ=1 as input and returns, as output, a combustion score for the material {(Sᵢ, Cᵢ), i=1, . . . , N}. The fuel optimization system (630) further includes an optimizer (637). The optimizer computes an optimal vector of N concentrations {Ĉᵢ, i=1, . . . , N} (625), that is a vector {Ĉᵢ, i=1, . . . , N} such that the Merit function applied to that vector is maximum, in a certain sense. In one or more embodiments, the value of the Merit function applied to {Ĉᵢ, i=1, . . . , N} is a global maximum of the Merit function. In other embodiments, the value of the Merit function applied to {Ĉᵢ, i=1, . . . , N} is an approximation of a global maximum of the Merit function. The list of physical properties (633), the Merit function (635) and the optimizer (637) are hosted on a computer (631), further included in the fuel optimization system (630). The computations involved by the Merit function (635) and optimizer (637) are run on the computer (631). The optimal vector of N concentrations {Ĉᵢ, i=1, . . . , N} (625) is returned to the fuel blending system (620), where a fuel (629) is produced as the material {(Sᵢ, Ĉᵢ), i=1, . . . , N} in the blending instrument (627), by blending the N substances (623) in the optimal concentrations given by the optimal vector of N concentrations (625). In one or more embodiments, the blending instrument (627) includes a storage tank, a pump, a plurality of valves and flow meters.

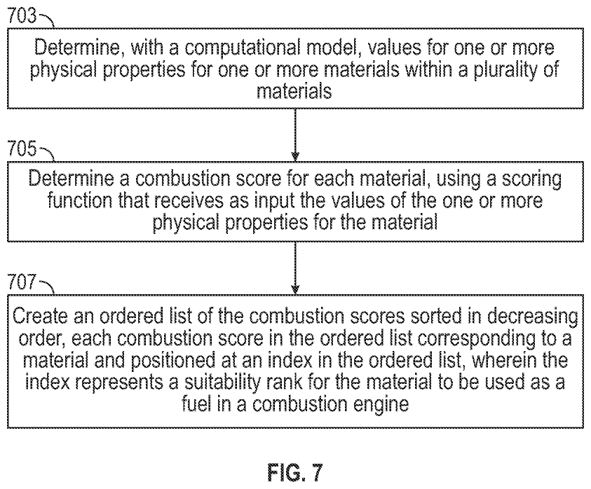

The flowchart in FIG. 7 depicts a method for comparing and ranking materials according to their combustion. In Step 703, values of some physical properties from a predefined set of physical properties for one or more materials are obtained, with a computational model. Throughout the description of FIG. 7, these physical properties, from the predefined set of physical properties, are referred to as “the physical properties”, and the term “physical property” stands for one of the physical properties. Any material in Step 703 may be composed of a single substance, or multiple substances. A material composed of multiple substances may be called a mixture.

Examples of materials in Step 703 include inorganic compounds, such as hydrogen, and organic compounds, such as gasoline, methane, propane, coal or ethanol. Organic compounds contain carbon in varying amounts and a given organic compound may be characterized by the number of carbon atoms that it contains. Notable examples of organic compounds that may be combusted as fuels and used as the materials in Step 703 include hydrocarbons, which are composed of only two elements: carbon and hydrogen. Hydrocarbons may be classified into various categories. Hydrocarbon mixtures may be classified and quantified by their Paraffins, Iso-paraffins, Olefins, Naphthenes, and Aromatics (PIONA) components. Paraffins are fully saturated compounds in that all of their carbon-carbon bonds are single bonds. A typical chemical formula for a paraffin is CₙH₂ₙ₊₂, where n is an integer. Examples of paraffins include methane CH₄ and octane C₈H₁₈. Iso-paraffins are isomeric forms of paraffins that have at least one branch in their carbon chain structure. They are also fully saturated and examples of chemical formulas for iso-paraffins are also CₙH₂ₙ₊₂, where n is an integer. Examples of iso-paraffins include isooctane C₈H₁₈ and isobutane C₄H₁₀. Olefins are unsaturated hydrocarbons in that they include one or more carbon-carbon double bonds. A typical chemical formula for an olefin is CₙH₂n, where n is an integer. Examples of olefins include ethene C₂H₄ and propene CH₃CHCH₂. Naphthenes are saturated hydrocarbons that have a cyclic carbon ring. A typical chemical formula for a naphthene is CₙH₂ₙ, where n is an integer. Examples of naphthenes include cyclopropane (CH₂)₃ and cyclohexane C₆H₁₂. Aromatics are unsaturated hydrocarbons that contain at least one benzene ring. An example of a chemical formula for an aromatic is C₄ₙ₊₂H₄ₙ₊₂, where n is an integer. Examples of aromatics include benzene C₆H₆ and Cyclotetradecaheptaene C₁₄H₁₄. Any molecule from the PIONA categories may be characterized by how many carbon atom it contains. For example, methane CH₄ is a C₁ paraffin, ethane C₂H₆ is a C₂ paraffin, and propane C₃H₈ is a C₃ paraffin. Examples of mixtures containing paraffins, such that the carbon number of each paraffin component is between 1 and 3 may be said to contain C₁-C₃ paraffins. An example of a material given by its PIONA components includes materials comprising C₅-C₇ paraffins, in an amount of 20% by volume of the composition, C₅-C₉ iso-paraffins, in an amount of 30% by volume of the composition, C₅-C₈ olefins in an amount of 20% by volume of the composition, C₅-C₁₀ naphthenes in an amount of 20% by volume of the composition and C₅-C₁₀ aromatics in an amount of 10% by volume of the composition.

In some embodiments, any material in Step 703 may further include oxygenates. Oxygenates are a class of organic compounds that contain oxygen atoms in their chemical structure, such as alcohols, ethers, esters, aldehydes and ketones. An alcohol includes an oxygen-hydrogen bond OH in its structure. Examples of alcohols include methanol CH₃OH, ethanol C₂H₅OH, iso-propanol C₃H₈O, n-propanol C₃H₈O and tert-butanol (CH₃)₃COH. Ethers contain an oxygen atom that connects two alkyl or aryl groups and hence may be formulated as ROR′, where each of R and R′ is an alkyl or aryl group. Examples of ethers include dimethyl ether CH₃OCH₃, diethyl ether CH₃CH₂OCH₂CH₃, furan C₄H₄₀, tetrahydrofuran (CH₂)₄O, and other furan derivatives. Esters are organic compounds with a general structure of RCOOR′, where each of R and R′ is an alkyl or aryl group. Examples of esters include ethyl acetate CH₃COC₂H₅ and methyl salicylate ethyl acetate C₈H₈O₃. Aldehydes are organic compounds with a general structure RCHO, where the bond between H and O is a double bond, and R is any side chain. Examples of aldehydes include monomeric formaldehyde CH₂O and acetaldehyde CH₃CHO. Ketones are organic compounds with a general structure RCOR′, where the bond between C and O is a double bond, and both R and R′ are side chains. Examples of ketones include acetone (CH₃)₂CO and acetophenone C₆H₅COCH₃. A material in Step 703 that is intended to be added to an existing fuel may be called a fuel additive. In one or more embodiments, a fuel additive may be added to a fuel mixture in order to optimize a combustion of the fuel.

The computational model in Step 703 may take various forms. In one or more embodiments, the computational model includes a physical model that connects an input material to an output value of a physical property by using laws of physics. In one or more embodiments, the computational model in Step 703 uses artificial intelligence (AI). Examples of AI models that predict a physical property from an input material include unsupervised machine learning models. A description of some embodiments for the computational model in Step 703 is given in the description of FIG. 2. For brevity FIG. 7, the computational model in Step 703 receives one or more materials as input and returns, for each material, values of the physical properties of the material as output. In some embodiments, the computational model processes one material at a time, in that it receives one material as input and returns values of the physical properties of the material as output. In this case, the computational model may execute multiple times, at least one time for each input material. Furthermore, in some embodiments, the computational model may return, for each material, values for the physical properties in a single run, while in other embodiments, the computational model may only be able to determine values of a subset of one or more of the physical properties at a time, in which case, for each material, multiple runs of the computational model may be necessary, to determine values for all the physical properties for the material. Therefore, Step 703 includes several scenarios, in which the computational model runs one or more times, receives one material as input for each run or multiple materials as input for each run, and returns, as output, a value for a single physical property, values for a subset of physical properties, or values for all the physical properties.

In Step 705, a combustion score is obtained for each material, using a scoring function that receives the values of the physical properties from Step 703 as input. An example of such a scoring function is given by the scoring function (215) in FIG. 2. In one or more embodiments, the combustion score for a material is a real number, assessing a combustion quality, of the combustion of the material. The combustion quality defines how suitable a material is to be combusted for the purpose of using the method of FIG. 7. The combustion quality of the combustion of a material may be defined in several ways, depending on such purpose. In one or more embodiments, the combustion quality of the combustion is defined as depending on one or more combustion properties, including an efficiency of the combustion, a performance of the combustion, an emission of toxic or greenhouse gas exhausted from the combustion, or any combination thereof. In such scenarios, the scoring function includes one or more factors selected from the group consisting of an efficiency factor, an emissions factor, and a performance factor. In one or more embodiments, the combustion score increases with an increase of the efficiency, performance or emissions factors. Examples of scoring functions that may be used in Step 705 are described in some of the next paragraphs of this disclosure.

In Step 707, an ordered list is created by sorting the combustion scores in descending order. Hence a combustion score at a given index in the ordered list is greater than or equal to a combustion score at a latter index in the ordered list. In one or more embodiments, the fact that a combustion score at a former index in the ordered list is greater than or equal to a combustion score at a latter index in the ordered list is interpreted as the fact that the combustion of a material having the combustion score at the former index is of better combustion quality than the combustion of a material having the combustion score at the later index in the ordered list, in turn indicating that a material having the combustion score at the former index in the ordered list is better suited for combustion than a material having the combustion score at the latter index. Accordingly, for each material, a suitability rank for the material is defined as the index in the ordered list of the combustion score of the material. The suitability rank of a material defines a preference for the material to be used as a fuel, compared to the other materials. The lower the suitability rank for a material, the more suitable the material is to be used as a fuel. The most suitable material to be used as a fuel is the material of rank one, or any material having a combustion score equal to the combustion score at the first index in the ordered list. In some embodiments, referring again to FIG. 2, the scoring function is defined by EQ. 2 and a first material will have a better rank than a second material, if the first material is more efficient, more performant, or emit less toxic or greenhouse gases than the second material.

The materials used in the method of FIG. 7 may be of various types. A material may comprise only one substance, or several substances. In one or more embodiments, a suitability rank threshold is obtained and materials with a suitability rank of less than or equal to the suitability rank threshold are selected to be used as fuels. For example, using a suitability rank of one, only one material having the highest combustion score is selected to be used as a fuel. In a scenario in which the suitability rank threshold is five, five materials having the highest five combustion scores are selected to be used as fuels. In one or more embodiments, the materials ranked in the method of FIG. 7 are considered as additive to an existing fuel, with a goal of an additive being, when added to a fuel, to enhance the combustion quality of the fuel. In one or more embodiments, a screening threshold is selected, and the method of FIG. 7 is classified as “high throughput screening” when the number of materials that are ranked is greater than the screening threshold.

Databases of materials are pre-defined and available to the public. Examples of such databases include “General Data Base” databases, such as the GDB-11 database, the GDB-13 database and the GDB-17 database. The GDB-11 database includes between 26 million and 27 million materials composed of a single molecule of at most 11 atoms; the GDB-13 database includes between 976 and 978 million materials composed of a single molecule of at most 13 atoms; the GDB-17 include between 165 and 167 billion of materials composed of a single molecule of at most 17 atoms. If a screening threshold of 1 million is selected, ranking the materials from each of the GDB-11, GDB-13 or GDB-17 databases is qualified as high throughput screening. In some embodiments, all materials from the GDB-13 database are ranked using the method of FIG. 7, a suitability rank threshold of five is selected, and the five materials with highest combustion scores are selected as additives to an existing fuel. In some embodiments, the five additives with highest combustion scores are alcohols with less than 5 carbon atoms.

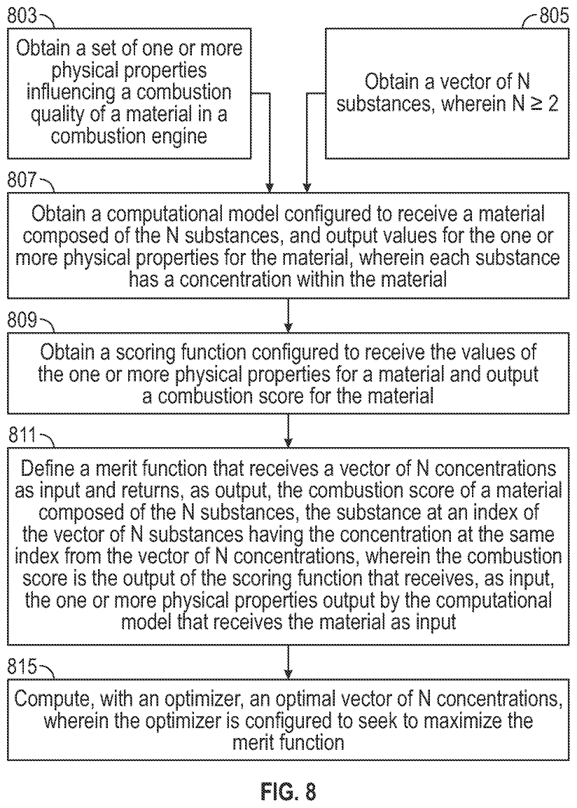

FIG. 8 depicts a method for optimizing concentrations of substances composing a material, in accordance with one or more embodiments. In Step 803, a set of physical properties is obtained, that may influence combustion of a material. There are many ways of obtaining such physical properties and an example is given by the system in FIG. 3. Examples of physical properties include the boiling point (BP), adiabatic flame temperature (AFT), laminar flame speed (LFS), heat of vaporization (HOV), carbon to oxygen ratio (C/O), research octane number (RON), and motor octane number (MON). A set of N≥2 substances is obtained in Step 805, where N is an integer. There are many ways of obtaining the N substances. In one or more embodiments, the number N≥2 is selected as a random integer below a certain threshold. Then, in some embodiments, the N substances are selected randomly from a database of hydrocarbons and oxygenates. In other embodiments, substances from a database are assigned a combustion score, the combustion score for each substance defined as a numerical value that assesses the quality of the combustion of the substance. The higher the combustion score for a substance, the more suitable it is to be used as a fuel. Then, the N substances with highest scores are selected to be used in Step 805. An example of assigning a score to a substance is given in the description of FIG. 2, by using a scoring function (215) that may receive properties of a substance as input. An example of the scoring function is given by EQ. 2-EQ. 5. In accordance with regulations, some substances may be prohibited depending on the geographic location, in which case they should be removed from the database before selecting substances from a database to be used in Step 805. Examples of databases from which substances might be selected include “General Data Base” databases, such as the GDB-11 database, the GDB-13 database and the GDB-17 database. Any screening process to select the substances from a database, to be used in Step 805, such as assigning a combustion score to the N substances and selecting the substances based on their combustion score, may be referred to as “high throughput screening” if the database is large. In some embodiments, a database may be considered as large if the number of its elements is greater than a certain screening threshold.

It is noted that each substance within the N substances from step 805 is composed of one species of molecule, and that a material may be formed by mixing these N substances. The N substances being already selected, a material composed of the N substances are completely defined by the concentrations of the N substances within the material. In Step 807, a computational model is obtained, configured to receive a material as input, and returns values of the physical properties, from the set of physical properties from Step 803, for the material. Examples of computational models that may be used in Step 807 include the computational model (205) from FIG. 2. Hence, in one or more embodiments, the computational model in Step 807 may make use of AI. Specifically, the computational model may include the AI model (207), that determines values of the physical properties for each substance within the material, and the chemical model (211) that determines values of the physical properties of the material, given the values of the physical properties of each of the N substances within the material and the concentration of each of the N substances within the material. An example of the AI model (207) is given in the descriptions of FIG. 4. An example of the chemical model (211) is the linear mole fraction formula EQ. 1. Other examples of the chemical model (211) may include AI.

In Step 809, a scoring function is obtained, configured to receive values of the physical properties from Step 803, for a material, and output a combustion score for the material, the combustion score defined as a numerical value that assesses the combustion quality of the material. The higher the combustion score for a material, the more suitable it is to be used as a fuel. An example of the scoring function is given by EQ. 2, that includes three factors that are considered of significant importance in accordance with some industry standards, in accordance with one or more embodiments: an efficiency factor, an emissions factor and a performance factor. Examples of an efficiency factor, an emissions factor and a performance factor are given in EQ. 3-EQ. 5. It is noted that the scoring function in EQ. 2, and the factors in EQ. 3-EQ. 5 are given as examples only and should not be considered as limiting. Many other embodiments exist for the scoring function in Step 809 and therefore, one with ordinary skill in the art will recognize that any variation of the scoring function in Step 809 may be employed without departing from the scope of this disclosure.

In Step 811, a Merit function is obtained. The Merit function is configured to receive the values of N concentrations as input and return, as output, a combustion score for the material of the N substances with the N concentrations. The Merit function combines the computational model from Step 807 and the scoring function from Step 809 as follows. Since the N substances are obtained in Step 805, any material of the N substances is completely defined by the concentrations of the N substances. Denoting the N substances as Sᵢ, i=1, . . . , N, and given N concentrations Cᵢ, i=1, . . . , N, as input, such that Σ₁^{N} Cᵢ=1, a material is formed, denoted as {(Sᵢ, Cᵢ), i=1, . . . , N}, composed of the N substances Sᵢ, i=1, . . . , N, each substance Sᵢ having the concentration Cᵢ, for i=1, . . . , N. The computational model from Step 807 may be applied to the material {(Sᵢ, Cᵢ), i=1, . . . , N} and return values of the physical properties defined in Step 803 as output. Then, the scoring function from Step 809 may be applied to these values of the physical properties and return a combustion score for the material {(Sᵢ, Cᵢ), i=1, . . . , N}. Denoting G as the computational model from Step 807, and H as the scoring function from Step 809, the Merit function receives the set of concentrations {Cᵢ,i=1, . . . , N} as input, and outputs the combustion score for the material {(Sᵢ, Cᵢ), i=1, . . . , N} as:

$$\{ {C}_{i} , i = 1 , ... , N \} \to Merit ( \{ {C}_{i} , i = 1 , ... , N \} ) = H ( G ( \{ ( {S}_{i} , {C}_{i} ) , i = 1 , ... , N \} ) ) . \qquad EQ . 9$$

In Step 815, the Merit function from EQ. 9 is optimized with an optimizer 8 that seeks a vector of N concentrations Ct, i=1, . . . , N such that the Merit function in EQ. 9 is maximum, that is, a vector of N concentrations {Cᵢ*, i=1, . . . , N} such that Σ₁^{N} Cᵢ*=1 and

$$for all vector \{ {C}_{i} , i = 1 , ... , N \} such that {\sum }_{1}^{N} {C}_{i} = 1 , \\ Merit ( \{ {C}_{i}^{★} , i = 1 , ... , N \} ) \geq Merit ( \{ {C}_{i} , i = 1 , ... , N \} ) . \qquad EQ . 10$$

Finding such vector, {Cᵢ*, i=1, . . . , N}, that satisfies EQ. 10, is only possible in rare cases, i.e. in cases for which the Merit function has very specific forms. For instance, if the equation ∇Merit(C{Cᵢ, i=1, . . . , N})=0 can be solved analytically for {Cᵢ, i=1, . . . , N} such that Σ₁^{N} Cᵢ=1, and has a discrete number of solutions, then the vector {C, i=1, . . . , N} can be defined, in some cases, as a vector satisfying EQ. 10 for all vector {Cᵢ, i=1, . . . , N} such that Σ₁^{N} Cᵢ=1 and ∇Merit({Cᵢ, i=1, . . . , N})=0. In such scenarios, the optimizer consists of solving the equation ∇Merit(C {Cᵢ, i=1, . . . , N})=0 for {Cᵢ*, i=1, . . . , N} such that Σ₁^{N} Cᵢ=1, and selecting {Cᵢ*, i=1, . . . , N}, within the set of solutions, that satisfies EQ. 10 for all vector {Cᵢ, i=1, . . . , N} such that Σ₁^{N} Cᵢ=1 and ∇Merit({Cᵢ, i=1, . . . , N})=0.

In the general case, the optimization problem of finding concentrations Cᵢ*, i=1, . . . , N such that Σ₁^{N} Cᵢ*=1 and satisfying EQ. 10 is done in an approximate sense, by iterating an optimizer until a certain convergence criterion is reached. The optimizer produces a recurrent sequence, indexed by an integer iteration number q≥0, of vectors of N concentrations {Cᵢ^{q}, i=1, . . . , N} such that Σ₁^{N} Cᵢ^{q}. In one or more embodiments, the optimizer is first initialized with an initial vector of N concentrations, denoted as {Cᵢ⁰, i=1, . . . , N}, that may be obtained in many ways. In one or more embodiments, the N concentrations may be obtained as random numbers Cᵢ⁰, i=1, . . . , N, such that Σ₁^{N} Cᵢ⁰=1. In other embodiments, the N concentrations may be defined uniformly as

$${C}_{i}^{0} = \frac{1}{N} , i = 1 , ... , N .$$

From the initial vector of N concentrations, the optimizer defines the values of the N concentrations {Cᵢ^{q}, i=1, . . . , N}, at each iteration q≥1, only depending on the values of the N concentrations {Cᵢ^{s}, i=1, . . . , N}, for s<q. In one or more embodiments, the optimizer is defined such that the values of the N concentrations {Cᵢ^{q}, i=1, . . . , N}, at each iteration q, only depends on the values of the N concentrations {Cᵢ^{q-1}, i=1, . . . , N}. Intuitively, the goal of the optimizer is that the Merit function applied to one of the terms of the sequence of vectors, {Cᵢ^{q}, i=1, . . . , N}, at an iteration q*, namely, Merit({Cᵢ^{q*}, i=1, . . . , N}), is as large as possible. In one or more embodiments, the optimizer is defined such that the sequence of Merits, Merit({Cᵢ^{q}, i=1, . . . , N}) is an increasing sequence and then, iterating the optimizer always produces a vector of N concentrations with a larger Merit than the previous iteration. The optimizer runs for a certain number of iterations, Q≥1, called the maximum iteration number. In one or more embodiments, the maximum iteration number Q is pre-defined and the convergence criterion for the optimizer is that the iteration number is Q. The convergence criterion for the optimizer can be defined in many other ways. In other embodiments, the convergence criterion is noting that the distance |Merit({Cᵢ^{q}, i=1, . . . , N})−Merit({Cᵢ^{q-1}, i=1, . . . , N})| is less than a predefined threshold. If a convergence criterion is met at some iteration, the optimizer is said to have converged, and the optimizer process stops. Regardless of the definition of the convergence criterion, the maximum iteration number is reached when the convergence criterion is met and denoted as Q. An optimal vector of N concentrations, in the scope of this disclosure, can be defined in many ways, as a vector of N concentrations {Cᵢ^{q*}, i=1, . . . , N} such that Merit({Cᵢ^{q*}, i=1, . . . , N})≥Merit({{Cᵢ⁰, i=1, . . . , N}), for some index q* such that 1≤q*≤Q. In one or more embodiments, the optimal vector of N concentrations is defined as a vector of N concentrations {Cᵢ^{Q}, i=1, . . . , N}, that is, the last value obtained by the optimizer when the convergence criterion is met. In other embodiments, the optimal vector of N concentrations is defined as the vector of N concentrations {CCᵢ^{q*}, i=1, . . . , N}, obtained at some iteration q*, that maximizes the Merit in the following sense:
for all q such that 0≤q≤Q,

$$Merit ( \{ {C}_{i}^{{q}^{★}} , i = 1 , ... , N \} ) \geq Merit ( \{ \{ {C}_{i}^{q} , i = 1 , ... , N \} ) . \qquad EQ . 11$$

In one or more embodiments, the optimizer is based on a gradient ascent method. Examples of gradient ascent methods include, but are not limited to a batch gradient ascent, a mini-batch gradient ascent, a gradient ascent with momentum, an Adam method or RMSprop method. In other embodiments, the optimizer is a scanning technique, in which the vectors {Cᵢ^{q}, i=1, . . . , N}, for 0≤q≤Q, are selected empirically, according to a selection criterion, and the optimal vector of N concentrations is defined as the vector of N concentrations {Cᵢ^{q*}, i=1, . . . , N}, obtained at an index q* satisfying EQ. 11.

It is noted that the optimization problem of finding concentrations Cᵢ*, i=1, . . . , N such that Σ₁^{N} Cᵢ*=1 and satisfying EQ. 10 is a constrained optimization problem, since the constraint Σ₁^{N} Cᵢ=1 must hold true. In one or more embodiments, further constraints are imposed to the optimization problem of finding concentrations Cᵢ*, i=1, . . . , N such that Σ₁^{N} Cᵢ*=1 and satisfying EQ. 10. As an example, further constraints may be imposed by regulatory standards, such as the regulatory standard EN228. The regulatory standard EN228 imposes the volume concentrations of methanol, in a fuel composition, not to exceed 3%, the ethanol volume concentration not to exceed 10%, the iso-propanol volume concentration not to exceed 12%, the iso-butanol volume concentration not to exceed 15%, the tert-butanol volume concentration not to exceed 15%, the ethers volume concentrations with five or more carbon atoms not to exceed 22%, and the volume concentration of other oxygenates not to exceed 15%. Under the regulatory standard EN228, these constraints, on the volume concentrations of some oxygenates, are added to the optimization problem of finding concentrations Ct, i=1, . . . , N such that Σ₁^{N} Cᵢ*=1 and satisfying EQ. 10. Generally, all constraints from the optimization problem of finding concentrations Cᵢ*, i=1, . . . , N such that Σ₁^{N} Cᵢ*=1 and satisfying EQ. 10 are added to the construction of the optimizer, and each vector of N concentrations {Cᵢ^{q}, i=1, . . . , N} from the optimizer is built to satisfy the constraints. The optimizer, that intends to solve a constrained optimization problem, can be of various forms. Examples of the optimizer in Step 815 include, but are not limited to a simplex method, an interior point method, a Lagrange multipliers method, a branch and bound method, and a generalized reduced gradient (GRG) method. As an example, the generalized reduced gradient (GRG) method defines the optimizer as

$$\{ {C}_{i}^{q + 1} , i = 1 , ... , N \} = \{ {C}_{i}^{q} , i = 1 , ... , N \} + α ( ∇ {Merit}^{q} - {R}^{q} ) . \qquad EQ . 12$$

In EQ. 12, α is a step size, ∇Merit^{q} is the gradient of the Merit function, evaluated at vector {Cᵢ^{q}, i=1, . . . , N}, and R^{q} is a N-dimensional reduction vector that forces the update {Cᵢ^{q+1}, i=1, . . . , N} to satisfy the constraints.

Referring again to FIG. 5, steps of the method shown in FIG. 8 may be used. Using the method of FIG. 8, a first optimal vector of N concentrations {C_{1,i}, i=1, . . . , N} (507) is computed for a vector of N substances (503) by following the steps 803-815 in FIG. 8, in which the scoring function obtained in Step 809 is called a first scoring function (505). A second optimal vector of N concentrations (511), {C_{2,i}, i=1, . . . , N}, is obtained by following the steps 803-815 in FIG. 8, using a second scoring function (509) obtained in Step 809 in FIG. 8, the second scoring function (509) being different from the first scoring function (505). Using the method of FIG. 8 with a third scoring function (513) in Step 809, a third optimal vector of N concentrations (515), {C_{3,i}, i=1, . . . , N}, is computed, and minimum and maximum blending requirements to form a material from the N substances from the vector of N substances (503) are determined as the N-dimensional vector of concentration ranges R (517) such that each component Rᵢ of the vector R is the range [min(C_{1,i}, C_{2,i}, C_{3,i}), max(C_{1,i}, C_{2,i}, C_{3,i})], for i=1, . . . , N. Extending the method further, assume that an integer number K of optimal vectors of N concentrations, {C_{k,i}, i=1, . . . , N} are obtained, for k=1, . . . , K, using the method described in FIG. 8, for K different scoring functions in Step 809 in FIG. 8. Also, in accordance with some embodiments, the N substances (623) in the system depicted in FIG. 6 are selected in the same fashion as Step 805 in FIG. 8. Also, embodiments for the optimizer described in Step 815 in FIG. 8 may be used as the optimizer 637 in FIG. 6.

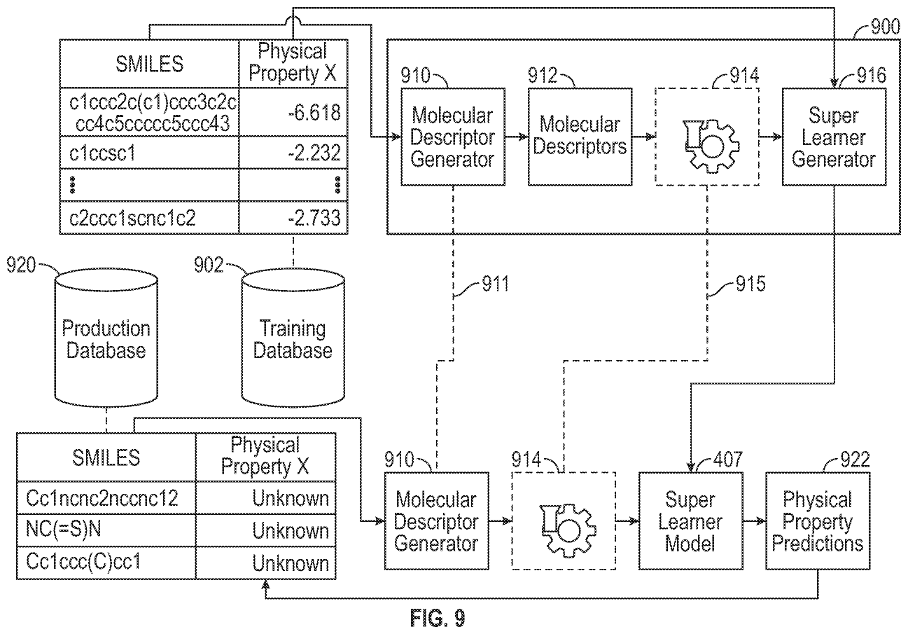

In accordance with one or more embodiments, FIG. 9 depicts a description of the super learner model (407) from FIG. 4. As stated, the super learner model (407) described herein is based on one or more machine-learned models. Machine-learning and a more detailed description of the super learner model are provided later in the instant disclosure. For now, it is noted that supervised machine-learned models require examples of input and associated output (i.e., target) pairs in order to learn a desired functional mapping. In one or more embodiments, an input is a simplified molecular-input line-entry system (SMILES) representation of a molecule and the output is a value of a physical property of the molecule. In one or more embodiments, the physical property of the molecule is a sensible or latent physical property or characteristic that can be determined through a measurement of the molecule in bulk or by evaluation of a system or environment of the molecule. That is, while the terms physical property or molecular physical property may be used herein, one with ordinary skill in the art will recognize that such a physical property is not limited to the description, behavior, or characterization of a single, individual molecule. Thus, the terms physical property and molecular physical property may be freely applied to a lump of matter made up of the molecule, known as substance. As an example, consider the molecule known as octane. Using this example, the input associated with octane is the SMILE representation of octane: 44 CC. As stated in previous paragraphs of this disclosure, examples for the associated output (value of a physical property) include a value for a boiling point (BP), an adiabatic flame temperature (AFT), a laminar flame speed (LFS), a heat of vaporization (HOV), a carbon to oxygen ratio (C/O), a research octane number (RON), and a motor octane number (MON). As will be described later, in the output of the super learner model (407) generated according to the methods and systems described herein may include more than one physical property.

In accordance with one or more embodiments, and as seen in FIG. 9, multiple input-target pairs are stored in a training database (902). The training database (902) includes, at a minimum, a SMILES representation and at least one associated physical property for each molecule listed in the training database (902). In FIG. 9, the single physical property is listed generically as physical property X. One with ordinary skill in the art will recognize that physical property X is a placeholder for any molecular physical property that can be tabulated (e.g., BP, AFT, LFS, etc.). In one or more embodiments, the training database (902) further includes values for additional properties, which may be generically listed here as physical property Y (not shown), physical property Z (not shown), etc. In one or more embodiments, the training database (902) may further include entry items such as a unique identification (ID) number, name, chemical formula, and/or description of each molecule.

Continuing with FIG. 9, a super learner generation system (900) is configured to receive the SMILES representation and physical property (physical property X) of the molecules listed in the training database (902). That is, the super learner generation system (900) is configured to receive data from the training database (902). In one or more embodiments, the super learner generation system (900) includes a molecular descriptor generator (910), a pre-processor (914), and a super learner generator (916). In one or more embodiments, the molecular descriptor generator (910) receives the SMILES representations of the molecules (one or more) in the training database (902) and produces a molecular descriptor (912) for each received molecule SMILES representation. The molecular descriptor (912) for each molecule is a vector (of at least length of one) that provides a numerical representation of the molecule. In one or more embodiments, the molecular descriptor generator (910) uses three numericalization techniques and concatenates the results into a single vector for each received SMILES representation. Specifically, in one or more embodiments, the molecular descriptor generator (910) converts a given SMILES representation of a molecule to a Morgan fingerprint, a set of 2D and 3D descriptor values, and an embedding vector.

The Morgan fingerprint is a reimplementation of the extended connectivity fingerprint (ECFP). Extended connectivity representations create fingerprints of varying lengths and depict circular atom neighborhoods. Generally, a variation accounts for each identifier as often as it appears in the molecule, rather than just once, to keep track of the frequency counts of the ECFP characteristics. This variant is frequently identified as ECFC. Some qualities of ECFPs are they can be calculated quickly, are not pre-defined and represent an infinite variety of different molecular features (including stereochemical information), and have features that indicate the presence of specific substructures, making it easier to understand analysis results. The ECFP algorithm can be modified to generate various circular fingerprints optimized for other applications.

In one or more embodiments, the molecular descriptor generator (910) receives a given SMILES representation and produces a bit representation of the molecule known herein as a Morgan fingerprint. The number of bits used in the Morgan fingerprint is determined and supplied by a user. In one or more embodiments, the Morgan fingerprint uses 2048 bits. As such, in one or more embodiments, the molecular descriptor generator (910) further includes one or more configuration parameters, wherein the configuration parameters dictate the behavior of the molecular descriptor generator (910). For example, the number of bits used by the Morgan fingerprint is a configuration parameter of the molecular descriptor generator (910). In one or more embodiments, the Morgan fingerprint for each molecule in the training database (902) is determined using an open-source software package such as RDKit, which may be readily imported into a Python programming environment.

Descriptor values are numerical values used to quantitatively describe the physical and chemical information of a molecule. A descriptor value must possess the following qualities: 1) invariance concerning labeling and numbering of atoms; 2) invariance to roto-translation; 3) an unambiguous algorithmically computable definition; and 4) values in a suitable numerical range for the set of molecules. Many types of descriptor values exist and can generally be classified as one of: 0D (count descriptors and so on); 1D (list of structural fragments, fingerprints); 2D (graph invariants); 3D (3D-MORSE descriptors, etc.); and 4D descriptor values (Volsurf, etc.). In one or more embodiments, the molecular descriptor generator (910) uses an open-source descriptor value calculation software, referenced herein as the Mordred package, to covert a SMILES representation of molecule to a set of 2D and/or 3D descriptor values. In one or more embodiments, the Mordred package is used to convert each SMILES representation in the training database (902) into a set of 1613 2D descriptor values.

An embedding vector is a numerical description of a molecule inspired by the art of natural language processing (NLP). In one or more embodiments, the molecular descriptor generator (910) uses an open-source embedding package known as Mol2Vec to convert a SMILES into an embedding vector. Mol2Vec molecular substructures derived from the Morgan fingerprinting algorithm as “words” and compounds as “sentences”. Thus, Mol2Vec learns molecular substructure's vector representations that point in similar directions for chemically related substructures. By adding the vectors of the individual substructures, molecules can be described as embedding vectors.

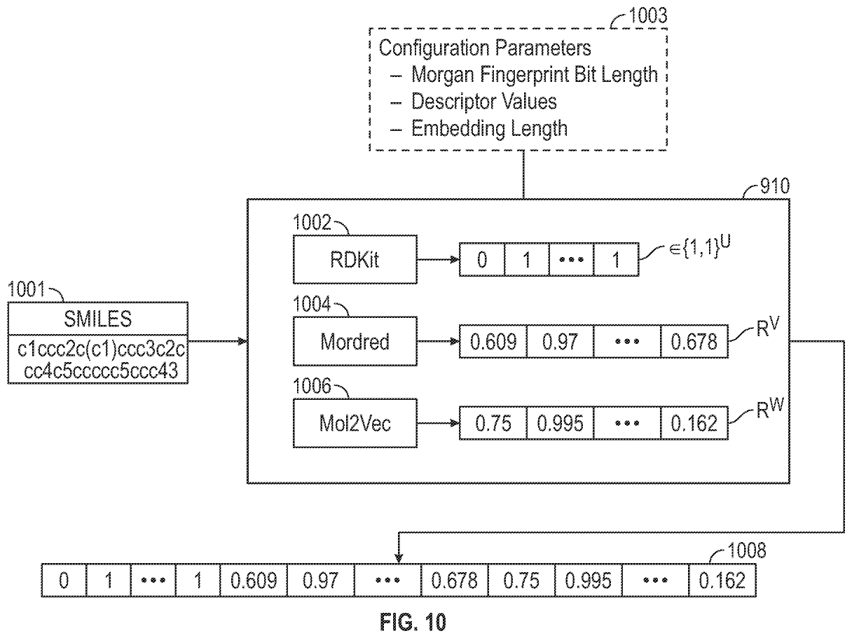

FIG. 10 depicts an example of the molecular descriptor generator (910) applied to an example SMILES representation (1001) of a molecule in accordance with one or more embodiments. As seen in the example of FIG. 10, the molecular descriptor generator (910) accepts a SMILES representation of a molecule, such as the example SMILES representation (1001) and processes the SMILES representation with RDKit (1002), Mordred (1004), and Mol2Vec (1006). RDKit (1002) processes, or otherwise converts, the example SMILES representation (1001) into a Morgan fingerprint representation. In one or more embodiments, the Morgan fingerprint representation is a bit vector of length U, where U is an integer greater than or equal to 1. Mordred (1004) processes, or otherwise converts, the example SMILES representation (1001) into a vector of descriptor values. In one or more embodiments, the descriptor values output by Mordred (1004) are defined by a user-provided set. In other embodiments, the descriptor values are defined by type, for example, 2D descriptor values. Herein, the number of descriptor values output by Mordred (1004) for a SMILES representation is V. Mol2Vec processes, or otherwise converts, the example SMILES representation (1001) to an embedding vector. The embedding vector has a length of W. In one or more embodiments, the molecular descriptor generator (910) is parameterized by, or has its behavior controlled by, configuration parameters (1003). The configuration parameters (1003) may define the number of bits used in the Morgan fingerprint, the number or type of descriptor values produced by Mordred (1004), and/or the length of the embedding vector. That is, the configuration parameters (1003) may directly, or indirectly, affect or define the values of U,V, and W. The molecular descriptor generator (910), for a given SMILES representation, concatenates the output of RDKit (1002), Mordred (1004), and Mol2Vec (1006) to form a molecular descriptor (912) for the associated molecule. For a given molecule, the length of the molecular descriptor (912) output by the molecular descriptor generator (910) is U+V+W.

FIG. 10 depicts an example molecular descriptor (1008) as output by the molecular descriptor generator (910) upon processing the example SMILES representation (1001) according to the configuration parameters (1003). In one or more embodiments, the configuration parameters (1003) are defined and set by a user. In other embodiments, the configuration parameters (1003) are learned while training the machine-learned models of the super learner model. Training of machine-learned models will be described in greater detail later in the instant disclosure.

Returning to FIG. 9, the molecular descriptor generator (910) is used to processes SMILES representations from the training database (902) to produce molecular descriptors (912) (one for each molecule represented in the training database). The molecular descriptors (912) undergo pre-processing by a pre-processor (914). Pre-processing may include identifying molecular descriptors (912) with missing or invalid values, performing an imputation procedure to populate missing or invalid values, or removing elements of molecular descriptor (912) across the entire dataset of molecular descriptors (912). Pre-processing may further include normalizing the molecular descriptors (912). The aforementioned pre-processing steps are associated with pre-processing parameters. For example, in the case of normalization, the pre-processing parameters include information about the means and standard deviations (or variances) applied to enact the normalization. Likewise, determined imputation values and identification of elements to be removed are also, if applicable, are also stored as pre-processing parameters. Thus, it may be said that the pre-processor (914) includes, or is configured by, a set of pre-processing parameters. In one or more embodiments, the pre-preprocessing parameters are defined by a user. In other embodiments, the pre-processing parameters are learned while training the machine-learned models of the super learner model (to be described in greater detail below).

Upon generating molecular descriptors (912) from the SMILES in the training database (902), and pre-processing the molecular descriptors (912), the pre-processed molecular descriptors (912) are passed with to a super learner generator (916) along with values of the physical property, or properties, of the molecules contained in the training database (902). That is, for a given molecule in the training database (902), the super learner generation system (900) forms an input-target pair consisting of the pre-processed molecular descriptor (912) (input) and value of the physical property (or properties) (target). The input-target pairs are used by the super learner generator (916) to produce a super learner model (407). The processes of the super learner generator (916) are described in greater detail later in the instant disclosure. For now, it is sufficient to say that the super learner model (407) is configured to accept a pre-processed molecular descriptor (912) of a molecule and output a prediction for values of one or more properties of the molecule. In the example of FIG. 9, physical property X is provided to the super learner generator (916) when forming input-target pair for each molecule in the training database (902).

Continuing with FIG. 9, the super learner model (407) generated by the super learner generator (916) of the super learner generation system (900) is used in production, that is, for determining physical properties of substances composing materials in the method of FIG. 7 and system in FIG. 2. The production setting consists of using the super learner model (407) to predict the value for the physical property (or values for physical properties) contained in the training database (902) (e.g., physical property X) for molecules contained in (stored/represented in) a production database (920). The production database (920), similarly to the training database (902), lists (or tabulates) SMILES representation of molecules of substances composing the materials in FIGS. 7 and 2. In contrast to the training database (902), the production database (920) does not include physical property information for any of its listed molecules. In one or more embodiments, the production database (920) may include a null entry, or other placeholder value, for the physical property (or properties) of its listed molecules. FIG. 9 depicts example contents of a production database (920). As seen in FIG. 9, the example production database (920) contains SMILES representations for three molecules but physical property X for these molecules is unknown (a placeholder value). In one or more embodiments, the production database (920) is a “General Data Base” databases, such as the GDB-11 database, the GDB-13 database and the GDB-17 database.

In production, the SMILES representation of one or more molecules listed in the production database (920) are processed with the molecular descriptor generator (910). It is important to note that any configuration parameters (1003) specified, or learned, when using the molecular descriptor generator (910) in the super learner generation system (900) are used in the production setting. The sharing of configuration parameters (1003) between the super learner generation system (900) and the use of the molecular descriptor generator (910) in the production setting is demonstrated by the first connection (911) in FIG. 9.

As depicted in FIG. 9, upon processing the SMILES representation of one or more molecules in the production database (920) with the molecular descriptor generator (910), the resulting molecular descriptors are pre-processed with the pre-processor (914). As stated, the pre-processor may include various pre-processing parameters defined, or learned, by the super learner generation system (900). These pre-processing parameters are used by the pre-processor (914) when pre-processing the molecules of the production database (920) as indicated by the second connection (915) in FIG. 9.

After pre-processing, the resulting pre-processed molecular descriptors of one or more molecules in the production database (920) are processed by the super learner model (407). Recall that the super learner model (407) was generated by the super learner generation system (900) operating on the training database (902). The super learner model (407) outputs a value of a physical property prediction for each received pre-processed molecular descriptor. In one or more embodiments, the physical property predictions (922) are stored in the production database (920) alongside their corresponding molecule. Thus, the super learner model (407) can predict a value of a physical property of a molecule given a pre-processed molecular descriptor of the molecule, where the pre-processed molecular descriptor is determined from only the SMILES representation of the molecule. As such, methods and systems described herein allow for physical property estimation of molecules, for which the physical property of the molecules is unknown, given only a SMILES representation of the molecules.

As stated, the computational model used in this disclosure, for example in the method of FIG. 7, the method of FIG. 8, and computational model (205) in the system described in FIG. 2, may include artificial intelligence (AI), which in some embodiments include the AI model (207). In FIG. 4 and FIG. 9, the AI model is the super learner model (407), composed of one or more machine-learned models. Furthermore, in accordance with some embodiments, the computational model may include a chemical model, such as the chemical model (211) in FIG. 2, which may also make use of AI. Artificial intelligence, broadly defined, is the extraction of patterns and insights from data. The phrases “artificial intelligence,” “machine learning,” “deep learning,” and “pattern recognition” are often convoluted, interchanged, and used synonymously throughout the literature. This ambiguity arises because the field of “extracting patterns and insights from data” was developed simultaneously and disjointedly among a number of classical arts like mathematics, statistics, and computer science. For consistency, the term artificial intelligence (AI) will be adopted herein, however, one skilled in the art will recognize that the concepts and methods detailed hereafter are not limited by this choice of nomenclature.

AI model types may include, but are not limited to, generalized linear models, Bayesian regression, random forests, and deep models such as neural networks, convolutional neural networks, and recurrent neural networks. AI model types, whether they are considered deep or not, are usually associated with additional “hyperparameters” which further describe the model. For example, hyperparameters providing further detail about a neural network may include, but are not limited to, the number of layers in the neural network, choice of activation functions, inclusion of batch normalization layers, and regularization strength. Commonly, in the literature, the selection of hyperparameters surrounding an AI model is referred to as selecting the model “architecture.” Once an AI model type and hyperparameters have been selected, the AI model is trained to perform a task.

A brief discussion and summary of various machine-learned model types is provided herein. However, one with ordinary skill in the art will recognize that a full discussion of every type of machine-learned model applicable to the methods and systems disclosed herein is not possible nor required to describe the super learner generation system (900) and use of the super learner model (407). Consequently, the following discussion of machine-learned models is provided by way of introduction to the art of machine-learning and does not impose a limitation on the present disclosure.

A notable example of an AI model that may be included in the computational model (205), super learner model (407) or chemical model (211) is a neural network (NN), such as a convolutional neural network (CNN) or a recurrent neural network (RNN). A cursory introduction to a NN is provided herein. However, it is noted that many variations of a NN exist. Therefore, one with ordinary skill in the art will recognize that any variation of the NN (or any other AI model) may be employed without departing from the scope of this disclosure. Further, it is emphasized that the following discussions of a NN is a basic summary and should not be considered limiting.

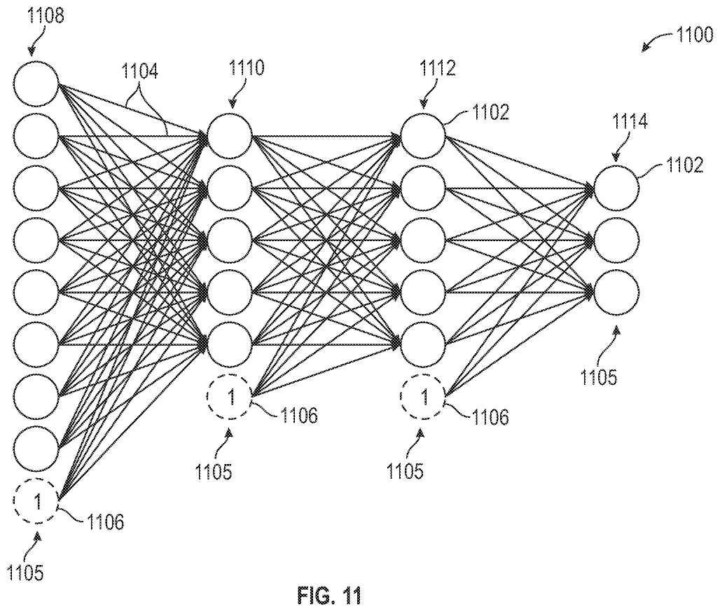

A diagram of a neural network is shown in FIG. 11. At a high level, a neural network (1100) may be graphically depicted as being composed of nodes (1102), where here any circle represents a node, and edges (1104), shown here as directed lines. The nodes (1102) may be grouped to form layers (1105). FIG. 11 displays four layers (1108, 1110, 1112, 1114) of nodes (1102) where the nodes (1102) are grouped into columns, however, the grouping need not be as shown in FIG. 11. The edges (1104) connect the nodes (1102). Edges (1104) may connect, or not connect, to any node(s) (1102) regardless of which layer (1105) the node(s) (1102) is in. That is, the nodes (1102) may be sparsely and residually connected. A neural network (1100) will have at least two layers (1105), where the first layer (1108) is considered the “input layer” and the last layer (1114) is the “output layer.” Any intermediate layer (1110, 1112) is usually described as a “hidden layer.” A neural network (1100) may have zero or more hidden layers (1110, 1112) and a neural network (1100) with at least one hidden layer (1110, 1112) may be described as a “deep” neural network or as a “deep learning method.” In general, a neural network (1100) may have more than one node (1102) in the output layer (1114). In this case the neural network (1100) may be referred to as a “multi-target” or “multi-output” network.

Nodes (1102) and edges (1104) carry additional associations. Namely, every edge is associated with a numerical value. The edge numerical values, or even the edges (1104) themselves, are often referred to as “weights” or “parameters.” While training a neural network (1100), numerical values are assigned to each edge (1104). Additionally, every node (1102) is associated with a numerical variable and an activation function. Activation functions are not limited to any functional class, but traditionally follow the form

$$A = f ( {\sum }_{i ∈ ( incoming )} [ {( node value )}_{i} {( edge value )}_{i} ] ) , \qquad EQ . 13$$

where i is an index that spans the set of “incoming” nodes (1102) and edges (1104) and ƒ is a user-defined function. Incoming nodes (1102) are those that, when the neural network (1100) is viewed or depicted as a directed graph (as in FIG. 11), have directed arrows that point to the node (1102) where the numerical value is being computed. Some functions for ƒ may include the linear function ƒ(x)=x, sigmoid function

$$f ( x ) = \frac{1}{1 + {e}^{- x}} ,$$

and rectified linear unit function ƒ(x)=max(0,x), however, many additional functions are commonly employed. Every node (1102) in a neural network (1100) may have a different associated activation function. Often, as a shorthand, activation functions are described by the function ƒ by which it is composed. That is, an activation function composed of a linear function ƒ may simply be referred to as a linear activation function without undue ambiguity.

When the neural network (1100) receives an input, the input is propagated through the network according to the activation functions and incoming node (1102) values and edge (1104) values to compute a value for each node (1102). That is, the numerical value for each node (1102) may change for each received input. Occasionally, nodes (1102) are assigned fixed numerical values, such as the value of 1, that are not affected by the input or altered according to edge (1104) values and activation functions. Fixed nodes (1102) are often referred to as “biases” or “bias nodes” (1106), displayed in FIG. 11 with a dashed circle.

In some implementations, the neural network (1100) may contain specialized layers (1105), such as a normalization layer, or additional connection procedures, like concatenation. One skilled in the art will appreciate that these alterations do not exceed the scope of this disclosure.

As noted, the training procedure for the neural network (1100) comprises assigning values to the edges (1104). To begin training the edges (1104) are assigned initial values. These values may be assigned randomly, assigned according to a prescribed distribution, assigned manually, or by some other assignment mechanism. Once edge (1104) values have been initialized, the neural network (1100) may act as a function, such that it may receive inputs and produce an output. As such, at least one input is propagated through the neural network (1100) to produce an output. Training data is provided to the neural network (1100). Generally, training data consists of pairs of inputs and associated targets. The targets represent the “ground truth,” or the otherwise desired output, upon processing the inputs. In the context of the super learner model (407), an input is a SMILES representation of a molecule in a substance and an output, or target, is the set of substance values for the physical properties (209). During training, the neural network (1100) processes at least one input from the training data and produces at least one output. Each neural network (1100) output is compared to its associated input data target. The comparison of the neural network (1100) output to the target is typically performed by a so-called “loss function;” although other names for this comparison function such as “error function,” “misfit function,” and “cost function” are commonly employed. Many types of loss functions are available, such as the mean-squared-error function, however, the general characteristic of a loss function is that the loss function provides a numerical evaluation of the similarity between the neural network (1100) output and the associated target. The loss function may also be constructed to impose additional constraints on the values assumed by the edges (1104), for example, by adding a penalty term, which may be physics-based, or a regularization term. Generally, the goal of a training procedure is to alter the edge (1104) values to promote similarity between the neural network (1100) output and associated target over the training data. Thus, the loss function is used to guide changes made to the edge (1104) values, typically through a process called “backpropagation”.

While a full review of the backpropagation process exceeds the scope of this disclosure, a brief summary is provided. Backpropagation consists of computing the gradient of the loss function over the edge (1104) values. The gradient indicates the direction of change in the edge (1104) values that results in the greatest change to the loss function. Because the gradient is local to the current edge (1104) values, the edge (1104) values are typically updated by a “step” in the direction indicated by the gradient. The step size is often referred to as the “learning rate” and need not remain fixed during the training process. Additionally, the step size and direction may be informed by previously seen edge (1104) values or previously computed gradients. Such methods for determining the step direction are usually referred to as “momentum” based methods.

Once the edge (1104) values have been updated, or altered from their initial values, through a backpropagation step, the neural network (1100) will likely produce different outputs. Thus, the procedure of propagating at least one input through the neural network (1100), comparing the neural network (1100) output with the associated target with a loss function, computing the gradient of the loss function with respect to the edge (1104) values, and updating the edge (1104) values with a step guided by the gradient, is repeated until a termination criterion is reached. Common termination criteria are: reaching a fixed number of edge (1104) updates, otherwise known as an iteration counter; a diminishing learning rate; noting no appreciable change in the loss function between iterations; reaching a specified performance metric as evaluated on the data or a separate hold-out data set. Once the termination criterion is satisfied, and the edge (1104) values are no longer intended to be altered, the neural network (1100) is said to be “trained”.

A structural grouping, or group, of weights is herein referred to as a “filter.” The number of weights in a filter is typically much less than the number of inputs. In a CNN, the filters can be thought as “sliding” over, or convolving with, the inputs to form an intermediate output or intermediate representation of the inputs which still possesses a structural relationship. Like unto the neural network (1100), the intermediate outputs are often further processed with an activation function. Many filters may be applied to the inputs to form many intermediate representations. Additional filters may be formed to operate on the intermediate representations creating more intermediate representations. This process may be repeated as prescribed by a user. There is a “final” group of intermediate representations, wherein no more filters act on these intermediate representations. In some instances, the structural relationship of the final intermediate representations is ablated; a process known as “flattening.” The flattened representation may be passed to a neural network (1100) to produce a final output. Note, that in this context, the neural network (1100) is still considered part of the CNN. Like unto a neural network (1100), a CNN is trained, after initialization of the filter weights, and the edge (1104) values of the neural network (1100), if present, with the backpropagation process in accordance with a loss function.

Another example of a machine-learned model type is a decision tree. As will be described, decisions trees often act as components, or sub-models, to other types of machine-learned models such as random forests and gradient boosted machines. A decision tree is composed of nodes. A decision is made at each node such that data present at the node are segmented. Typically, at each node, the data at said node, are split into two parts, or segmented bimodally, however, multimodal segmentation is possible. The segmented data can be considered another node and may be further segmented. As such, a decision tree represents a sequence of segmentation rules. The segmentation rule (or decision) at each node is determined by an evaluation process. The evaluation process usually involves calculating which segmentation scheme results in the greatest homogeneity or reduction in variance in the segmented data. However, a detailed description of this evaluation process, or other potential segmentation scheme selection methods, is omitted for brevity and does not limit the scope of the present disclosure.

Further, if at a node in a decision tree, the data are no longer to be segmented, that node is said to be a “leaf node.” Commonly, values of data found within a leaf node are aggregated, or further modeled, such as by a linear model, so that a leaf node represents a class or an aggregated value (e.g., an average). A decision tree can be configured in a variety of ways, such as, but not limited to, choosing the segmentation scheme evaluation process, limiting the number of segmentations, and limiting the number of leaf nodes. Generally, when the number of segmentations or leaf nodes in a decision tree is limited, the decision tree is said to be a “weak learner”.

Commonly, gradient boosted machine models are constructed using decision trees. Hereafter, a gradient boosted machine model using decision trees is referred to as a gradient boosted trees model. In most implementations, the decision trees from which a gradient boosted trees model is composed are weak learners. In a gradient boosted trees model, the decision trees are ensembled in series, wherein each decision tree makes a weighted adjustment to the output of the preceding decision trees in the series. The process of forming an ensemble of decision trees in series, and making weighted adjustments, to form a gradient boosted trees model is best illustrated by considering the training process of a gradient boosted trees model.

Training a gradient boosted trees model consists of the selection of segmentation rules for each node in each decision tree; that is, training each decision tree. Once trained, a decision tree is capable of processing data. For example, a decision tree may receive a data input (e.g., a pre-processed molecular descriptor). The data input is sequentially transferred to nodes within the decision tree according to the segmentation rules of the decision tree. Once the data input is transferred to a leaf node, the decision tree outputs the assigned class or aggregate value (e.g., molecular property value) of the associated leaf node.

Generally, training a gradient boosted model firstly consists of making a simple prediction (SP) for the target data (i.e., the one or more molecular properties). The simple prediction (SP) may be the average property value over the training database (902). The simple prediction (SP) is subtracted from the targets to form a first residuals. The first decision tree in the series is created and trained, wherein the first decision tree attempts to predict the first residuals forming first residual predictions. The first residual predictions from the first decision tree are scaled by a scaling parameter. In the context of gradient boosted trees the scaling parameter is known as the “learning rate” (η). The learning rate is one of the hyperparameters governing the behavior of the gradient boosted trees model. The learning rate (n) may be fixed for all decision trees or may be variable or adaptive. The first residual predictions of the first decision tree are multiplied by the learning rate (n) and added to the simple prediction (SP) to form first predictions. The first predictions are subtracted from the targets to form second residuals. A second decision tree is created and trained using the data inputs and the second residuals as targets such that it produces second residual predictions. The second residual predictions are multiplied by the learning rate (n) and are added to the first predictions forming second predictions. This process is repeated recursively until a termination criterion is achieved.

Many termination criteria exist and are not all enumerated here for brevity. Common termination criteria are terminating training when a pre-defined number of decision trees has been reached, or when improvement in the residuals is no longer observed.

Once trained, a gradient boosted trees model may make predictions using input data. To do so, the input data is passed to each decision tree, which will form a plurality of residual predictions. The plurality of residual predictions is multiplied by the learning rate (n), summed across every decision tree, and added to the simple prediction (SP) formed during training to produce the gradient boosted trees predictions.

One with ordinary skill in the art will appreciate that many adaptions may be made to gradient boosted trees models and that these adaptions do not exceed the scope of this disclosure. Some adaptions may be algorithmic optimizations, efficient handling of sparse data, use of out-of-core computing, and parallelization for distributed computing. Commonly, when such adaptions are applied to a gradient boosted trees model, the model is known in the literature as XGBoost.

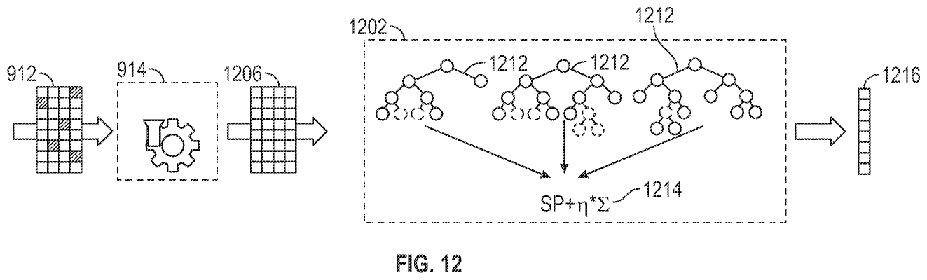

FIG. 12 depicts, generally, the flow of data through a trained gradient boosted trees model (1202) in accordance with one or more embodiments. For concision, in FIG. 12, it is assumed that one or more SMILES representations have been previously processed by the molecular descriptor generator (910). Thus, the processes of FIG. 12 begin with a set of one or more molecular descriptors (912) as input data to the gradient boosted trees model (1202). The molecular descriptors (912) are pre-processed by the pre-processor (914) as previously described. The result of the pre-processing is pre-processed molecular descriptors (1206).

The pre-processed molecular descriptors (1206) are passed to the gradient boosted trees model (1202) composed of a plurality of decision trees (1212). As such, the pre-processed molecular descriptors (1206) are processed by each of the plurality of decision trees (1212) and the output of each decision tree is collected, multiplied by the learning rate (n), summed, and added to the simple prediction (SP) established during training forming an ensemble (1214). The result of the ensemble (1214) is returned as the gradient boosted trees model prediction (1216). In the context of the current disclosure, the gradient boosted trees model prediction (1216) is the physical property predictions (922).

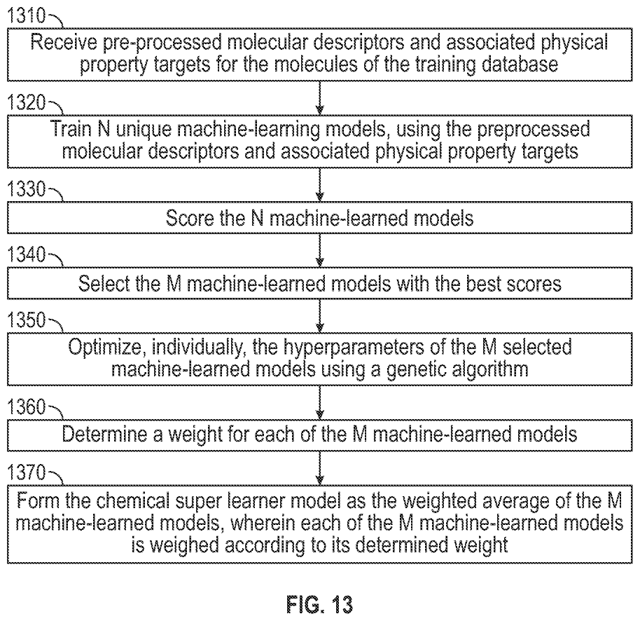

With an introduction to machine-learning and some machine-learned model types presented, the processes of the super learner generator (916) are described in greater detail here. Turning to FIG. 13, FIG. 13 depicts a flowchart outlining the processes of the super learner generator (916) in accordance with one or more embodiments. As depicted in Block 1310 of FIG. 13, the super learner generator (916) receives pre-processed molecular descriptors and associated target physical properties for the molecules of the training database (902). Each molecule may be associated with one or more physical properties. Further, each molecule in the database is represented with a SMILES representation that is processed by the molecular descriptor generator (910) and pre-processor (914) to produce the pre-processed molecular descriptors.

In one or more embodiments, the pre-processed molecular descriptors are split into various sets such as a training set, validation set, and testing set for training, scoring, and generalization error estimation purposes. Because data splitting is a common practice when training, evaluating, and testing a machine-learned model, it is not explicitly depicted in FIG. 13. One with ordinary skill in the art will recognize that any data splitting technique may be applied to the pre-processed molecular descriptors without departing from the scope of this disclosure. In one or more embodiments, the molecules of the training database (902) are split using a molecular scaffold splitting technique such that the molecules in any of the resulting data partitions are structurally dissimilar. In one or more embodiments, the molecules of the training database (902) are split into one or more datasets before being processed by the molecular descriptor generator (910) and/or pre-processor (914) such that the configuration parameters and/or pre-processor parameters may be learned in training according to a training dataset. Thus, it is understood that training, evaluation, and optimization an any machine-learned models discussed below may be according to one or more pre-defined partitions of the training database (902).

In Block 1320, N unique machine-learned models are trained independently using the pre-processed molecular descriptors and associated physical property targets, where N is an integer greater than or equal to 1. In one or more embodiments, the N machine-learned models are trained using a cross-validation scheme applied to a training dataset (or partition) of the pre-processed molecular descriptors. In one or more embodiments, 40 machine-learned models are trained, where the type, sub-type, and shorthand name of each model is represented in Table I.

TABLE I
The type, subtype, model, and shorthand model name of 40 machine-learned models
trained using pre-processed molecular descriptors and target physical properties
in accordance with one or more embodiments of the instant disclosure.

| | | S | | Model |
|---|---|---|---|---|
| Type | Sub-type | No | Model | name |
| Linear models | Classical | 1 | LinearRegression | LR |
| | | 2 | Ridge | RR |
| | | 3 | RidgeCV | RCVR |
| | | 4 | SGDRegressor | SGDR |
| | Regressors | 5 | ElasticNet | ENR |
| | with variable | 6 | ElasticNetCV | ENCVR |
| | selection | 7 | Lars | LaR |
| | | 8 | LarsCV | LaCVR |
| | | 9 | Lasso | LasR |
| | | 10 | LassoCV | LasCVR |
| | | 11 | LassoLars | LLR |
| | | 12 | LassoLarsIC | LLICR |
| | | 13 | LassoLarsCV | LLCVR |
| | | 14 | OrthogonalMatchingPursuit | OMPR |
| | | 15 | OrthogonalMatchingPursuitCV | OMPCVR |
| | Bayesian | 16 | BayesianRidge | BRR |
| | regressors | | | |
| | Outlier-robust | 17 | RANSACRegressor | RANR |
| | regressors | 18 | HuberRegressor | HR |
| | Generalized | 19 | PoissonRegressor | PR |
| | linear models | 20 | GammaRegressor | GR |
| | (GLM) for | 21 | TweedieRegressor | TR |
| | regression | | | |
| | Miscellaneous | 22 | PassiveAggressiveRegressor | PAR |
| Kernel Ridge models | Kernel Ridge | 23 | KernelRidge | KRR |
| | Regression | | | |
| Gaussian Processes | Gaussian | 24 | GaussianProcessRegressor | GPR |
| | Processes | | | |
| Nearest Neighbors | Nearest | 25 | KNeighborsRegressor | KNR |
| | Neighbors | | | |
| Neural network models | Neural network | 26 | MLPRegressor | MLPR |
| | models | | | |
| Support Vector Machines | Support Vector | 27 | SVR | SVR |
| | Machines | 28 | LinearSVR | LSVR |
| | | 29 | NuSVR | NSVR |
| Decision Trees | Classical | 30 | DecisionTreeRegressor | DTR |
| | | 31 | ExtraTreeRegressor | ETR |
| | Ensemble | 32 | ExtraTreesRegressor | ETsR |
| | Methods | 33 | RandomForestRegressor | RFR |
| | | 34 | AdaBoostRegressor | ABR |
| | | 35 | BaggingRegressor | BRR |
| | | 36 | GradientBoostingRegressor | GBR |
| | | 37 | HistGradientBoostingRegressor | HGBR |
| | External | 38 | XGBRegressor | XGBR |
| | | 39 | LGBMRegressor | LGBMR |
| | | 40 | CatBoostRegressor | CBR |

Continuing, with FIG. 13, once trained, the N machine-learned models are scored. A model's score represents the ability of the model to accurately predict the target physical property of a molecule given the pre-processed molecular descriptor of the molecule. Thus, the score of the model is computed by comparing the physical property predictions produced by the model to the true (known) physical property targets. Any scoring or comparison function known in the art may be used, including but not limited to: mean absolute error (MAE), mean square error (MSE), root mean square error (RMSE) and coefficient of determination (R²), defined as:

$$MAE = \frac{1}{n} {\sum }_{i = 1}^{i = n} \vert {\hat{y}}_{i} - {y}_{i} \vert , \qquad EQ . 11$$

$$MSE = \frac{1}{n} {\sum }_{i = 1}^{i = n} {( {\hat{y}}_{i} - {y}_{i} )}^{2} , \qquad EQ . 12$$

$$RMSE = \sqrt{\frac{1}{n} {\sum }_{i = 1}^{i = n} {( {\hat{y}}_{i} - {y}_{i} )}^{2}} , \qquad EQ . 13$$

$${R}^{2} = 1 - \frac{{\sum }_{i = 1}^{i = n} {( {\hat{y}}_{i} - {y}_{i} )}^{2}}{{\sum }_{i = 1}^{i = n} {( {y}_{i} - \overline{y} )}^{2}} . \qquad EQ . 14$$

In EQ. 11, EQ. 12, EQ. 13 and EQ. 14, n denotes the number of training examples, each training example being defined as an input-output pair, (xᵢ, yᵢ), for i=1, . . . , n, in which xᵢ is the input and the true physical property target yᵢ is the output,

$$\overline{y} = \frac{1}{n} {\sum }_{i = 1}^{i = n} {y}_{i} ,$$

and ŷᵢ denotes the value of the physical property prediction of the super learner model receiving xᵢ as input, for i=1, . . . , n. Further, scoring may take place using a cross-validation scheme or across a partition of the training database (902) not used during training such as a validation dataset and/or test dataset.

Once scored, in Block 1340 the M top scoring machine-learned models are selected for further use, where M is an integer and 1≤M≤N. In Block 1350 the hyperparameters of each of the M selected machine-learned models are optimized. The hyperparameters of the M models are optimized independent from each other. In one or more embodiments, the hyperparameter optimization is guided by an evaluation score determined using a cross-validation scheme over a training dataset. In other embodiments, the hyperparameter optimization is guided by an evaluation score over a validation set. In accordance with one or more embodiments, the hyperparameter optimization is performed using a genetic algorithm. It is noted that the order of Blocks 1340 and 1350 may be readily interchanged without departing from the scope of this disclosure. That is, in some embodiments, the hyperparameters of all N machine-learned models are optimized (or tuned) and then the top M performing models are selected. However, the embodiment depicted in FIG. 13 is generally preferred as hyperparameter optimization can be a computationally expensive task such that it is beneficial to only optimized the hyperparameters of M machine-learned models where M≤N.

After hyperparameter optimization the M selected models are combined to form the super learner model (407). The combination is performed according to a weighted average. Specifically, if ψ represents one of the M selected machine-learned models, then the super learner model (407), Ψ_{SL}, is given by

$${Ψ}_{SL} = {\sum }_{m = 1}^{M} {w}_{m} {ψ}_{m} , {w}_{m} \geq 0 , \qquad EQ . 15$$

where wₘ represents the weight associated with the m^{th} machine-learned model. To ensure that the M selected machine-learned models are combined according to a proper weighted average, EQ. 15 is subject to the following constraint,

$${\sum }_{m = 1}^{M} {w}_{m} = 1. \qquad EQ . 16$$

Thus, to form the super learner model (407), for each of the M selected machine-learned models, a weight wₘ must be determined. In Block 1360, a weight is determined for each of the M machine-learned models. In one or more embodiments, the weights are determined using a Sequential Least-Squares Programming method (SLSQP) to include the equality constraint of EQ. 16. Specifically, the SLSQP method determines the weights for the super learner model (407), Ψ_{SL}, that result in the highest performing (i.e., scoring) model when evaluated on a validation set and/or test set of the molecules of the training database (902). Once the weights of the super learner model (407) have been determined according to Block 1360, the super learner model (407) is officially formed as the weighted average of the M selected machine-learned models (EQ. 15) as depicted in Block 1370.

While the various blocks in FIG. 13 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

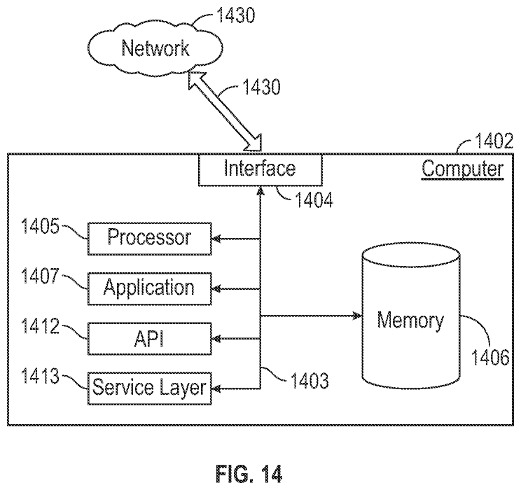

The computations mentioned in this disclosure may be performed by a computer, such as the computer (631) in FIG. 6. In that regard, FIG. 14 depicts a block diagram of a computer (1402) used to provide computational functionalities associated with described algorithms, methods, functions, processes, flows, and procedures as described in this disclosure, according to one or more embodiments. The illustrated computer (1402) is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer (1402) may include a computer that includes an input device, such as a keypad, keyboard, touch screen, or other device that can accept user information, and an output device that conveys information associated with the operation of the computer (1402), including digital data, visual, or audio information (or a combination of information), or a GUI.

The computer (1402) can serve in a role as a client, network component, a server, a database or other persistency, or any other component (or a combination of roles) of a computer system for performing the subject matter described in the instant disclosure. In some implementations, one or more components of the computer (1402) may be configured to operate within environments, including cloud-computing-based, local, global, or other environments (or a combination of environments).

At a high level, the computer (1402) is an electronic computing device operable to receive, transmit, process, store, or manage data and information associated with the described subject matter. According to some implementations, the computer (1402) may also include or be communicably coupled with an application server, e-mail server, web server, caching server, streaming data server, business intelligence (BI) server, or other server (or a combination of servers).

The computer (1402) can receive requests over network (1430) from a client application (for example, executing on another computer (1402) and responding to the received requests by processing the said requests in an appropriate software application. In addition, requests may also be sent to the computer (1402) from internal users (for example, from a command console or by other appropriate access method), external or third-parties, other automated applications, as well as any other appropriate entities, individuals, systems, or computers.

Each of the components of the computer (1402) can communicate using a system bus (1403). In some implementations, any or all of the components of the computer (1402), both hardware or software (or a combination of hardware and software), may interface with each other or the interface (1404) (or a combination of both) over the system bus (1403) using an application programming interface (API) (1412) or a service layer (1413) (or a combination of the API (1412) and service layer (1413). The API (1412) may include specifications for routines, data structures, and object classes. The API (1412) may be either computer-language independent or dependent and refer to a complete interface, a single function, or even a set of APIs. The service layer (1413) provides software services to the computer (1402) or other components (whether or not illustrated) that are communicably coupled to the computer (1402). The functionality of the computer (1402) may be accessible for all service consumers using this service layer. Software services, such as those provided by the service layer (1413), provide reusable, defined business functionalities through a defined interface. For example, the interface may be software written in JAVA, C++, or other suitable language providing data in extensible markup language (XML) format or another suitable format. While illustrated as an integrated component of the computer (1402), alternative implementations may illustrate the API (1412) or the service layer (1413) as stand-alone components in relation to other components of the computer (1402) or other components (whether or not illustrated) that are communicably coupled to the computer (1402). Moreover, any or all parts of the API (1412) or the service layer (1413) may be implemented as children or sub-modules of another software module, enterprise application, or hardware module without departing from the scope of this disclosure.

The computer (1402) includes an interface (1404). Although illustrated as a single interface (1404) in FIG. 14, two or more interfaces (1404) may be used according to particular needs, desires, or particular implementations of the computer (1402). The interface (1404) is used by the computer (1402) for communicating with other systems in a distributed environment that are connected to the network (1430). Generally, the interface (1404) includes logic encoded in software or hardware (or a combination of software and hardware) and operable to communicate with the network (1430). More specifically, the interface (1404) may include software supporting one or more communication protocols associated with communications such that the network (1430) or interface's hardware is operable to communicate physical signals within and outside of the illustrated computer (1402).

The computer (1402) includes at least one computer processor (1405). Although illustrated as a single computer processor (1405) in FIG. 14, two or more processors may be used according to particular needs, desires, or particular implementations of the computer (1402). Generally, the computer processor (1405) executes instructions and manipulates data to perform the operations of the computer (1402) and any algorithms, methods, functions, processes, flows, and procedures as described in the instant disclosure.

The computer (1402) also includes a memory (1406) that holds data for the computer (1402) or other components (or a combination of both) that can be connected to the network (1430). The memory may be a non-transitory computer readable medium. For example, memory (1406) can be a database storing data consistent with this disclosure. Although illustrated as a single memory (1406) in FIG. 14, two or more memories may be used according to particular needs, desires, or particular implementations of the computer (1402) and the described functionality. While memory (1406) is illustrated as an integral component of the computer (1402), in alternative implementations, memory (1406) can be external to the computer (1402).

The application (1407) is an algorithmic software engine providing functionality according to particular needs, desires, or particular implementations of the computer (1402), particularly with respect to functionality described in this disclosure. For example, the application (1407) can serve as one or more components, modules, applications, etc. Further, although illustrated as a single application (1407), the application (1407) may be implemented as multiple applications (1407) on the computer (1402). In addition, although illustrated as integral to the computer (1402), in alternative implementations, the application (1407) can be external to the computer (1402).

There may be any number of computers such as the computer (1402) associated with, or external to, a computer system containing the computer (1402), wherein each computer (1402) communicates over network (1430). Further, the term “client,” “user,” and other appropriate terminology may be used interchangeably as appropriate without departing from the scope of this disclosure. Moreover, this disclosure contemplates that many users may use one computer (1402), or that one user may use multiple computers such as the computer (1402).

The following examples are merely illustrative and should not be interpreted as limiting the scope of the present disclosure.

### EXAMPLES

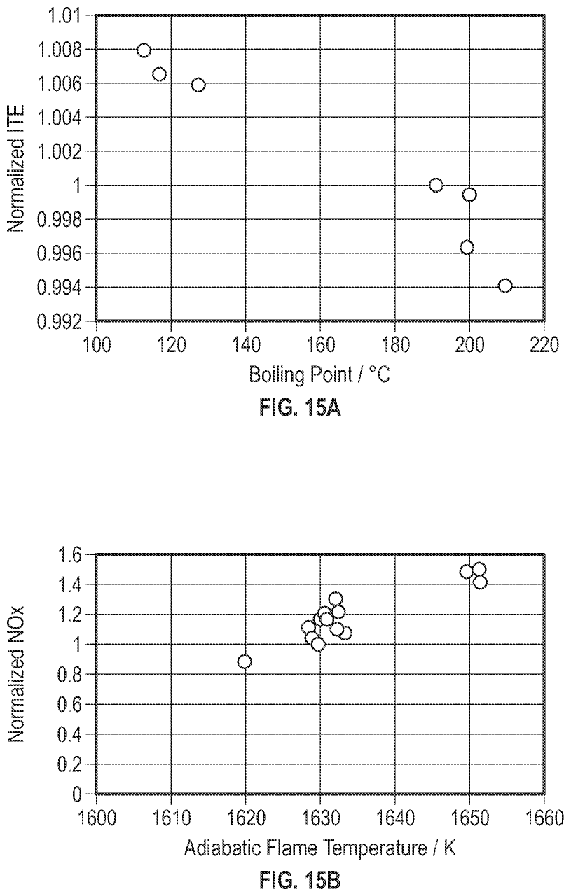

FIG. 15A is a scattered plot of results of an experiment. The experiment includes combusting a certain number N≥1 of substances denoted as Sᵢ, for i=1, . . . , N whose boiling points (BP) are known, and measuring a normalized indicated thermal efficiency (ITE) of the combustion each substance. In the scattered plot in FIG. 15A, the abscissa xᵢ of each point (xᵢ, yᵢ) represents the value of the BP of a substance Sᵢ, and the ordinate yᵢ of the point (xᵢ, yᵢ) is the value of the measured normalized ITE of the combustion of the substance through the experiment. In one or more embodiments, the BP is used as the master property (303) in FIG. 3, the normalized ITE is used as the combustion property (305) in FIG. 3, in order to determine whether the BP might be used as a physical property within the set of physical properties via the inclusion action (309) in FIG. 3. The statistic (307) in FIG. 3 is then computed by using the values of (xᵢ, yᵢ), for i=1, . . . , N, obtained in this experiment. In some embodiments, the statistic (307) is a correlation computed from EQ. 6, EQ. 7 and EQ. 8. In one or more embodiments, an interpretation of the scattered plot in FIG. 15A is that the BP of a chemical substance is linearly correlated with its normalized ITE, and the BP should be included as a physical property within the set of physical properties through the inclusion action (309) in FIG. 3.

FIG. 15B is a scattered plot of results of an experiment. The experiment includes combusting a certain number N≥1 of substances denoted as Sᵢ, for i=1, . . . , N whose adiabatic flame temperatures (AFT) are known, and measuring a normalized NOₓ emission of the combustion each substance. In the scattered plot in FIG. 15B, the abscissa xᵢ of each point (xᵢ, yᵢ) represents the value of the AFT of a substance Sᵢ, and the ordinate yt of the point (xᵢ, yᵢ) is the value of the measured normalized NOₓ emission of the combustion of the substance through the experiment. In one or more embodiments, the AFT is used as the master property (303) in FIG. 3, the NOₓ emission is used as the combustion property (305) in FIG. 3, in order to determine whether the AFT might be used as a physical property within the set of physical properties via the inclusion action (309) in FIG. 3. The statistic (307) in FIG. 3 is then computed by using the values of (xᵢ, yᵢ), for i=1, . . . , N, obtained in this experiment. In some embodiments, the statistic (307) is a correlation computed from EQ. 6, EQ. 7 and EQ. 8. In one or more embodiments, an interpretation of the scattered plot in FIG. 15A is that the AFT of a chemical substance is linearly correlated with its NOₓ emission, and the AFT should be included as a physical property within the set of physical properties through the inclusion action (309) in FIG. 3.

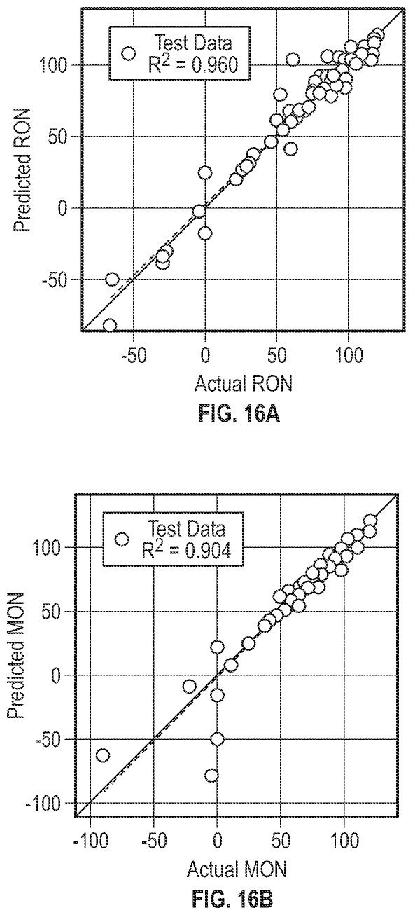

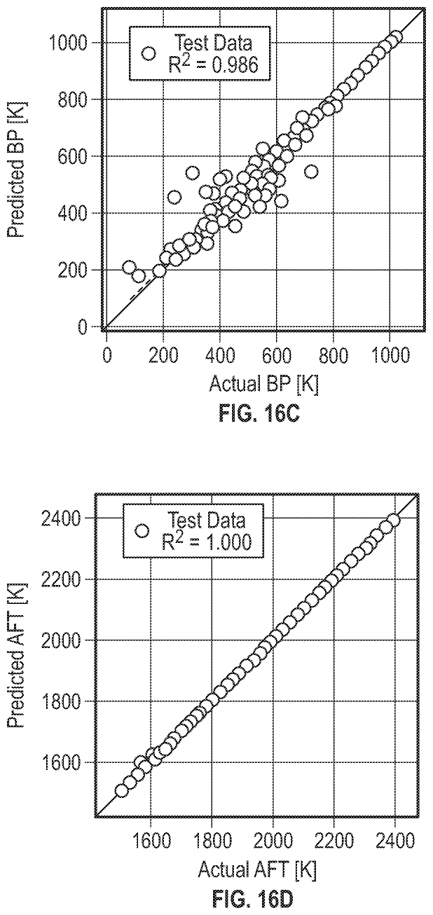

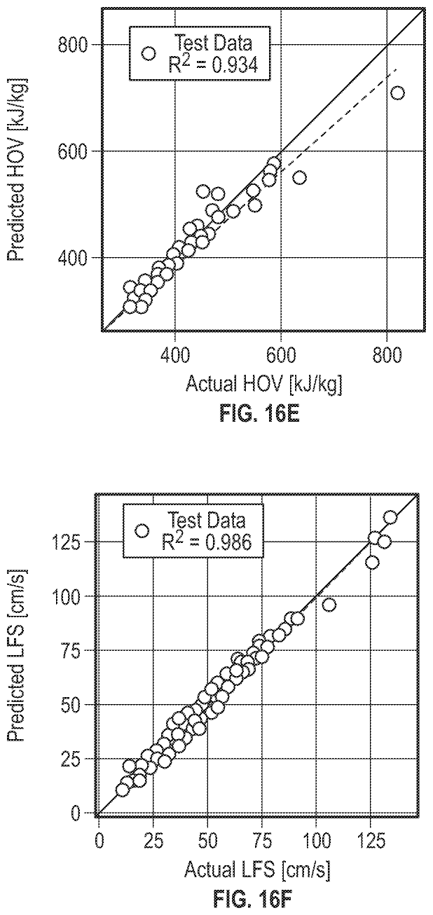

FIG. 16A includes a scattered plot representing a result of testing the super learner model (407) described in FIG. 4, FIG. 9 and FIG. 10. In FIG. 16A, each point (yᵢ, ŷᵢ) represents, for each substance within a certain number N≥1 of substances, denoted as Sᵢ, for i=1, . . . , N, the actual RON, yᵢ, of the substance, plotted against a value, ŷᵢ, of the RON of that substance predicted by the super learner model. The graph in FIG. 16A further includes a dash line, that represent the equation of the identity line ŷ=y. The graph in FIG. 16A further includes a value for the coefficient of determination (R²), as computed by using EQ. 14. If the super learner model performed perfectly, the points (yᵢ,ŷᵢ) would be located on the dashed line on the graph and the R² would be equal to 1. In one or more embodiments, the points (yᵢ,ŷᵢ) in FIG. 16A are considered to be located close to the dashed line, apart from some outliers, and the R² is considered to be close to 1. An interpretation of the points (yᵢ,ŷᵢ) being located close to the dashed line, apart from some outliers, and the R² being close to 1 is that the RONs y predicted by the super learner model are close to the actual RON yᵢ. According to one of more embodiments, an interpretation that the RONs ŷᵢ predicted by the super learner model are close to the actual RON yᵢ is that the super learner model may predict a RON of a substance accurately, for a substance for which the actual value of the RON is unknown.

FIG. 16B includes a scattered plot representing a result of testing the super learner model (407) described in FIG. 4, FIG. 9 and FIG. 10. In FIG. 16B, each point (yᵢ,ŷᵢ) represents, for each substance within a certain number N≥1 of substances, denoted as Sᵢ, for i=1, . . . , N, the actual MON, yᵢ, of the substance, plotted against a value, ŷᵢ, of the MON of that substance predicted by the super learner model. The graph in FIG. 16B further includes a dash line, that represent the equation of the identity line ŷ=y. The graph in FIG. 16B further includes a value for the coefficient of determination (R²), as computed by using EQ. 14. If the super learner model performed perfectly, the points (yᵢ,ŷᵢ) would be located on the dashed line on the graph and the R² would be equal to 1. In one or more embodiments, the points (yᵢ,ŷᵢ) in FIG. 16B are considered to be located close to the dashed line, apart from some outliers, and the R² is considered to be close to 1. An interpretation of the points (yᵢ,ŷᵢ) being located close to the dashed line, apart from some outliers, and the R² being close to 1 is that the MONs ŷᵢ predicted by the super learner model are close to the actual MON yᵢ. According to one of more embodiments, an interpretation that the MONs ŷᵢ predicted by the super learner model are close to the actual MON yᵢ is that the super learner model may predict a MON of a substance accurately, for a substance for which the actual value of the MON is unknown.

FIG. 16C includes a scattered plot representing a result of testing the super learner model (407) described in FIG. 4, FIG. 9 and FIG. 10. In FIG. 16C, each point (yᵢ,ŷᵢ) represents, for each substance within a certain number N≥1 of substances, denoted as Sᵢ, for i=1, . . . , N, the actual BP, yᵢ, of the substance, plotted against a value, ŷᵢ, of the BP of that substance predicted by the super learner model. The graph in FIG. 16C further includes a dash line, that represent the equation of the identity line ŷ=y. The graph in FIG. 16C further includes a value for the coefficient of determination (R²), as computed by using EQ. 14. If the super learner model performed perfectly, the points (yᵢ,ŷᵢ) would be located on the dashed line on the graph and the R² would be equal to 1. In one or more embodiments, the points (yᵢ,ŷᵢ) in FIG. 16C are considered to be located close to the dashed line, apart from some outliers, and the R² is considered to be close to 1. An interpretation of the points (yᵢ,ŷᵢ) being located close to the dashed line, apart from some outliers, and the R² being close to 1 is that the BPs ŷᵢ predicted by the super learner model are close to the actual BP yᵢ. According to one of more embodiments, an interpretation that the BPs ŷᵢ predicted by the super learner model are close to the actual BP yᵢ is that the super learner model may predict a BP of a substance accurately, for a substance for which the actual value of the BP is unknown.

FIG. 16D includes a scattered plot representing a result of testing the super learner model (407) described in FIG. 4, FIG. 9 and FIG. 10. In FIG. 16D, each point (yᵢ,ŷᵢ) represents, for each substance within a certain number N≥1 of substances, denoted as Sᵢ, for i=1, . . . , N, the actual AFT, yᵢ, of the substance, plotted against a value, ŷᵢ, of the AFT of that substance predicted by the super learner model. The graph in FIG. 16D further includes a dash line, that represent the equation of the identity line ŷ=y. The graph in FIG. 16D further includes a value for the coefficient of determination (R²), as computed by using EQ. 14. If the super learner model performed perfectly, the points (yᵢ,ŷᵢ) would be located on the dashed line on the graph and the R² would be equal to 1. In one or more embodiments, the points (yᵢ,ŷᵢ) in FIG. 16D are considered to be located close to the dashed line, apart from some outliers, and the R² is considered to be close to 1. An interpretation of the points (yᵢ,ŷᵢ) being located close to the dashed line, apart from some outliers, and the R² being close to 1 is that the AFTs ŷᵢ predicted by the super learner model are close to the actual AFT yᵢ. According to one of more embodiments, an interpretation that the AFTs ŷᵢ predicted by the super learner model are close to the actual AFT yᵢ is that the super learner model may predict a AFT of a substance accurately, for a substance for which the actual value of the AFT is unknown.

FIG. 16E includes a scattered plot representing a result of testing the super learner model (407) described in FIG. 4, FIG. 9 and FIG. 10. In FIG. 16E, each point (yᵢ,ŷᵢ) represents, for each substance within a certain number N≥1 of substances, denoted as Sᵢ, for i=1, . . . , N, the actual HOV, yᵢ, of the substance, plotted against a value, ŷᵢ, of the HOV of that substance predicted by the super learner model. The graph in FIG. 16E further includes a dash line, that represent the equation of the identity line ŷ=y. The graph in FIG. 16E further includes a value for the coefficient of determination (R²), as computed by using EQ. 14. If the super learner model performed perfectly, the points (yᵢ,ŷᵢ) would be located on the dashed line on the graph and the R² would be equal to 1. In one or more embodiments, the points (yᵢ,ŷᵢ) in FIG. 16E are considered to be located close to the dashed line, apart from some outliers, and the R² is considered to be close to 1. An interpretation of the points (yᵢ,ŷᵢ) being located close to the dashed line, apart from some outliers, and the R² being close to 1 is that the HOVs ŷᵢ predicted by the super learner model are close to the actual HOV yᵢ. According to one of more embodiments, an interpretation that the HOVs ŷᵢ predicted by the super learner model are close to the actual HOV yᵢ is that the super learner model may predict a HOV of a substance accurately, for a substance for which the actual value of the HOV is unknown.

FIG. 16F includes a scattered plot representing a result of testing the super learner model (407) described in FIG. 4, FIG. 9 and FIG. 10. In FIG. 16F, each point (yᵢ,ŷᵢ) represents, for each substance within a certain number N≥1 of substances, denoted as Sᵢ, for i=1, . . . , N, the actual LFS, yᵢ, of the substance, plotted against a value, y, of the LFS of that substance predicted by the super learner model. The graph in FIG. 16F further includes a dash line, that represent the equation of the identity line ŷ=y. The graph in FIG. 16F further includes a value for the coefficient of determination (R²), as computed by using EQ. 14. If the super learner model performed perfectly, the points (yᵢ,ŷᵢ) would be located on the dashed line on the graph and the R² would be equal to 1. In one or more embodiments, the points (yᵢ,ŷᵢ) in FIG. 16F are considered to be located close to the dashed line, apart from some outliers, and the R² is considered to be close to 1. An interpretation of the points (yᵢ,ŷᵢ) being located close to the dashed line, apart from some outliers, and the R² being close to 1 is that the LFSs ŷᵢ predicted by the super learner model are close to the actual LFS yᵢ. According to one of more embodiments, an interpretation that the LFSs ŷᵢ predicted by the super learner model are close to the actual LFS yᵢ is that the super learner model may predict a LFS of a substance accurately, for a substance for which the actual value of the LFS is unknown.

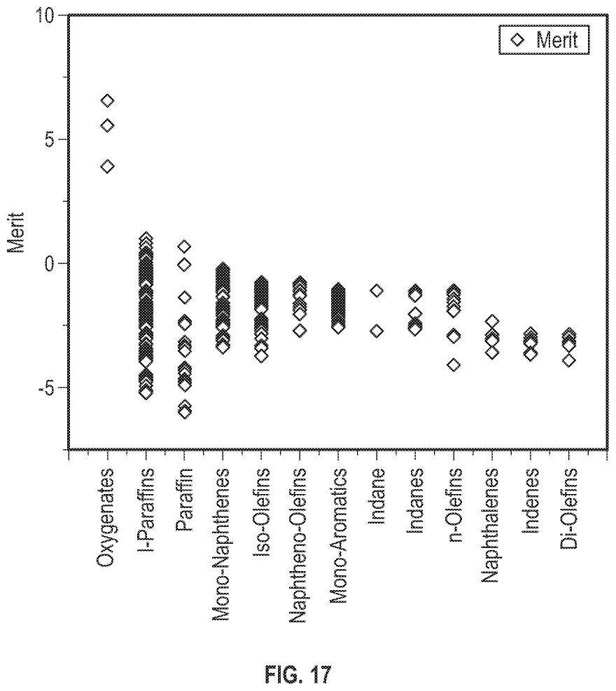

FIG. 17 depicts combustion scores for substances composing six fuels, obtained using the method described in FIG. 7. The substances are classified into thirteen categories, and a scattered plot of the combustion scores is displayed for each category. The thirteen categories are the oxygenates, iso-paraffins, paraffin, mono-naphthenes, iso-olefins, naphtheno-olefins, mono-aromatics, indane, indanes, n-olefins, naphthalenes, indenes, and di-olefins. The scoring function used in Step 705 of the method described in FIG. 7 is the scoring function in EQ. 2 with weights w₁=0.5, w₂=0.3 and w₃=0.2. The computational model used in Step 703 of the method described in FIG. 7 is the specific embodiment of the computational model (205) that includes the AI model (207) and chemical model (211). The AI model (207) that was used is the super learner model described in FIGS. 4-10 and 13.

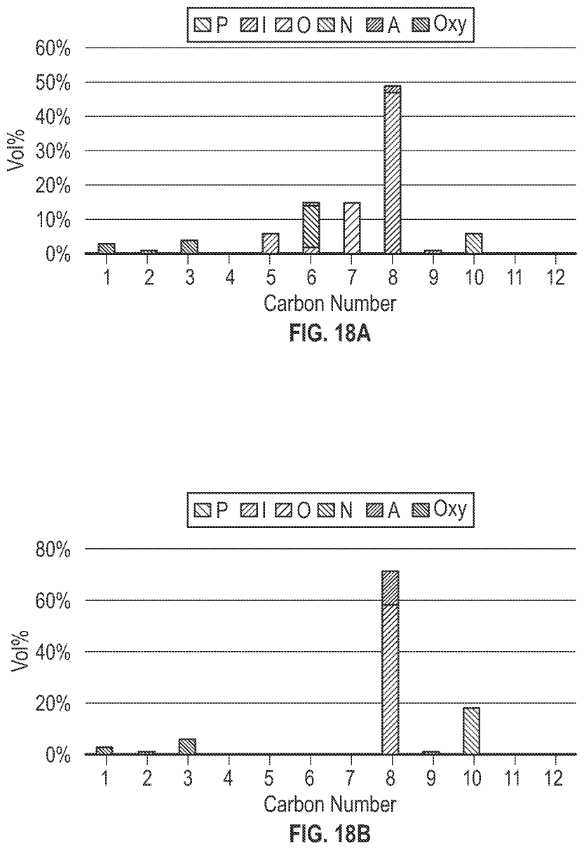

FIG. 18A depicts an example of a PIONA analysis of a fuel composition obtained by using the optimization method described in FIG. 8, that is, a fuel is composed of N substances, each substance having a concentration from the optimal set of concentrations in Step 815 in FIG. 8. The concentrations of the fuel composition in FIG. 18A were obtained by using the scoring function in EQ. 2 with weights w₁=0.5, w₂=0.3 and w₃=0.2, and the computational model (205) that includes the AI model (207) and chemical model (211). The AI model (207) that was used is the super learner model described in FIGS. 4-10 and 13. The optimization problem from EQ. 17 is constrained to meet the specifications EN228, which are defined in a previous paragraph. The volume concentrations of the paraffin, iso-paraffin, olefins, naphthenes, aromatics and oxygenates within the N substances of the fuel are plotted as a bar plot against carbon numbers of the N substances. The component with the highest concentration is the iso-paraffin component with a carbon number of 8. The component with the second highest concentration is the olefin component with a carbon number of 7. The component with the lowest concentration is the aromatic component with various carbon numbers.

FIG. 18B depicts an example of a PIONA analysis of a fuel composition obtained by using the optimization method described in FIG. 8, that is, a fuel is composed of N substances, each substance having a concentration from the optimal set of concentrations in Step 815 in FIG. 8. The concentrations of the fuel composition in FIG. 18B were obtained by using the scoring function in EQ. 2 with weights w₁=0.5, w₂=0.3 and w₃=0.2, and the computational model (205) that includes the AI model (207) and chemical model (211). The AI model (207) that was used is the super learner model described in FIGS. 4-10 and 13. The optimization problem from EQ. 17 is constrained to meet the specifications EN228, and an extra constraint that BP>110° C. The volume concentrations of the paraffin, iso-paraffin, olefins, naphthenes, aromatics and oxygenates within the N substances of the fuel are plotted as a bar plot against carbon numbers of the N substances. The component with the highest concentration is the iso-paraffin component with a carbon number of 8. The component with the second highest concentration is the naphthene component with a carbon number of 10. The aromatic component is higher than the one in FIG. 18A, especially the aromatic component with a carbon number of 8. The olefin component is reduced, compared to FIG. 18A.

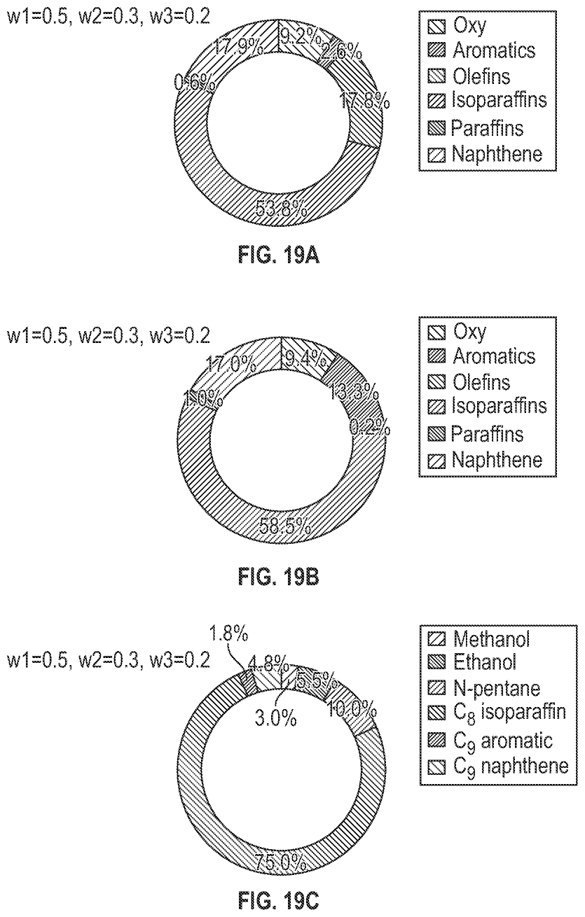

FIG. 19A depicts a pie chart of the same example of a PIONA analysis depicted as a bar chart in FIG. 18A, of a fuel composition obtained by using the optimization method described in FIG. 8, that is, a fuel is composed of N substances, each substance having a concentration from the optimal set of concentrations in Step 815 in FIG. 8. The concentrations of the fuel composition in FIG. 19A were obtained by using the scoring function in EQ. 2 with weights w₁=0.5, w₂=0.3 and w₃=0.2, and the computational model (205) that includes the AI model (207) and chemical model (211). The AI model (207) that was used is the super learner model described in FIGS. 4-10 and 13. The optimization problem from EQ. 17 is constrained to meet the specifications EN228, which are defined in a previous paragraph. The volume concentrations of the paraffin, iso-paraffin, olefins, naphthenes, aromatics and oxygenates within the N substances of the fuel are plotted as a pie chart. The component with the highest concentration is the iso-paraffin component with a carbon number of 8. The component with the second highest concentration is the olefin component with a carbon number of 7. The component with the lowest concentration is the aromatic component with various carbon numbers.

FIG. 19B depicts a pie chart of the same example of a PIONA analysis depicted as a bar chart in FIG. 18B, of a fuel composition obtained by using the optimization method described in FIG. 8, that is, a fuel is composed of N substances, each substance having a concentration from the optimal set of concentrations in Step 815 in FIG. 8. The concentrations of the fuel composition in FIG. 19B were obtained by using the scoring function in EQ. 2 with weights w₁=0.5, w₂=0.3 and w₃=0.2, and the computational model (205) that includes the AI model (207) and chemical model (211). The AI model (207) that was used is the super learner model described in FIGS. 4-10 and 13. The optimization problem from EQ. 17 is constrained to meet the specifications EN228, and an extra constraint that BP>110° C. The volume concentrations of the paraffin, iso-paraffin, olefins, naphthenes, aromatics and oxygenates within the N substances of the fuel are plotted as a pie chart. The component with the highest concentration is the iso-paraffin component with a carbon number of 8. The component with the second highest concentration is the naphthene component with a carbon number of 10. The aromatic component is higher than the one in FIG. 19A, especially the aromatic component with a carbon number of 8. The olefin component is reduced, compared to FIG. 19A.

FIG. 19C depicts a pie chart of a fuel composition obtained by using the optimization method described in FIG. 8. That is, a fuel is composed of N substances, each substance having a concentration from the optimal set of concentrations in Step 815 in FIG. 8. The concentrations of the fuel composition in FIG. 19C were obtained by using the scoring function in EQ. 2 with weights w₁=0.5, w₂=0.3 and w₃=0.2, and the computational model (205) that includes the AI model (207) and chemical model (211). The AI model (207) that was used is the super learner model described in FIGS. 4-10 and 13. The optimization problem from EQ. 17 is constrained to meet the specifications EN228, defined in a previous paragraph. The fuel composition in FIG. 19C is composed of C₈ iso-paraffins, C₉ naphthenes, C₉ aromatics, and the additives methanol, ethanol, and n-pentane, whose volume concentrations are plotted as a pie chart. The component with the highest concentration is the C₈ iso-paraffin. The component with the second highest concentration is the n-pentane. The component with the lowest concentration is the C₉ aromatics component.

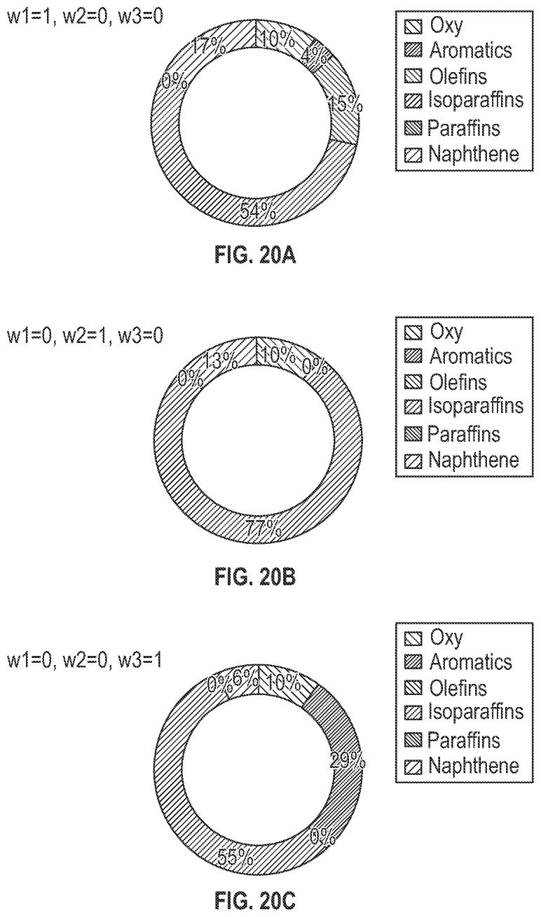

FIGS. 20A, 20B and 20C depict examples of PIONA analyses of fuel compositions obtained by using the optimization method described in FIG. 8, that is, a fuel is composed of N substances, each substance having a concentration from the optimal set of concentrations in Step 815 in FIG. 8. The computational model used in Step 807 of the method described in FIG. 8 is the specific embodiment of the computational model (205) that includes the AI model (207) and chemical model (211). The AI model (207) that was used is the super learner model described in FIGS. 4-13. The optimization problem from EQ. 17 is constrained to meet the specifications EN228, which are defined in a previous paragraph. The volume concentrations of the paraffin, iso-paraffin, olefins, naphthenes, aromatics and oxygenates within the N substances of the fuel are plotted as pie charts. The concentrations of the fuel composition in FIG. 20A were obtained by using the scoring function in EQ. 2 with weights w₁=1, w₂=0 and w₃=0, which means that only the efficiency factor is taken into account in EQ. 2. In FIG. 20A, the volume concentrations in the resulting fuel are 10% for the oxygenates, 4% for the aromatics, 15% for the olefins, 54% for the iso-paraffins, 0% for the paraffins and 17% for the naphthenes. The concentrations of the fuel composition in FIG. 20B were obtained by using the scoring function in EQ. 2 with weights w₁=0, w₂=1 and w₃=0, which means that only the emissions factor is taken into account in EQ. 2. In FIG. 20B, the volume concentrations in the resulting fuel are 10% for the oxygenates, 0% for the aromatics, 0% for the olefins, 77% for the iso-paraffins, 0% for the paraffins and 13% for the naphthenes. The concentrations of the fuel composition in FIG. 20C were obtained by using the scoring function in EQ. 2 with weights w₁=0, w₂=0 and w₃=1, which means that only the performance factor is taken into account in EQ. 2. In FIG. 20C, the volume concentrations in the resulting fuel are 10% for the oxygenates, 29% for the aromatics, 0% for the olefins, 55% for the iso-paraffins, 0% for the paraffins and 6% for the naphthenes.

To conduct the experiments leading to the examples in FIGS. 17-20C, the super learner model described in FIGS. 4-10 and 13 was trained for a plurality of physical properties. Table II includes values for the Metrics defined in EQ. 11, EQ. 12 and EQ. 14, computed for the testing of the super learner model described in FIGS. 4-13 for the physical properties: RON, MON, BP, AFT, HOV and LFS.

TABLE II
Metrics defined in EQ. 11, EQ. 12 and EQ. 14, computed for the
testing of the super learner model described in FIGS. 4-9 for
the physical properties: RON, MON, BP, AFT, HOV and LFS

| Property | Test R² | Test MAE | Test RMSE |
|---|---|---|---|
| RON | 0.960 | 3.559 | 6.870 |
| MON | 0.904 | 2.814 | 8.519 |
| BP | 0.986 | 7.204 | 8.622 |
| AFT | 1.000 | 1.722 | 3.334 |
| HOV | 0.934 | 12.419 | 21.380 |
| LFS | 0.986 | 1.277 | 1.824 |

Although only a few example embodiments have been described in detail above, those skilled in the art will readily appreciate that many modifications are possible in the example embodiments without materially departing from this invention. In addition, many modifications will be appreciated by those skilled in the art to adapt a particular instrument, situation, or material to embodiments of the disclosure without departing from the essential scope thereof. Accordingly, all such modifications are intended to be included within the scope of this disclosure as defined in the following claims.

Furthermore, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by one or more embodiments described herein. At the very least, and not as an attempt to limit the application of the doctrine of equivalents to the scope of the claim, each numerical parameter should at least be construed in light of the number of reported significant digits and by applying ordinary rounding techniques.

What is claimed:

1. A composition, comprising:
C₅-C₇ paraffins, in an amount not exceeding 20% by volume of the composition;
C₅-C₉ iso-paraffins, in an amount from 30% to 90% by volume of the composition;
C₅-C₈ olefins, in an amount not exceeding 40% by volume of the composition;
C₅-C₁₀ naphthenes, in an amount not exceeding 20% by volume of the composition;
C₅-C₁₀ aromatics, in an amount not exceeding 30% by volume of the composition; and
a fuel additive comprising C₁-C₅ oxygenates, in an amount from 1% to 15% by volume of the composition.

2. The composition of claim 1, wherein the composition has activity as a fuel for a combustion engine.

3. The composition of claim 2, wherein the combustion engine is an ultra-lean burn engine.

4. The composition of claim 1, wherein the C₅-C₈ olefins comprise:
C₅-C₇ n-olefins, in an amount not exceeding 20% by volume of the composition; and
C₅-C₈ Iso-olefins, in an amount not exceeding 20% by volume of the composition.

5. The composition of claim 1, wherein the fuel additive comprises an alcohol.

6. The composition of claim 5, where the alcohol is selected from the group consisting of methanol, ethanol, iso-propanol, n-propanol, tert-butanol and combinations thereof.

7. The composition of claim 1, wherein the fuel additive comprises the C₁-C₅ oxygenates in respective amounts not more than as allowed by a regulatory standard.

8. A method, comprising:
determining, using a computational model, values for one or more physical properties for each material within a plurality of materials;
determining a combustion score for each material, using a scoring function that receives as input the values of the one or more physical properties for the material; and
creating an ordered list of the combustion scores sorted in decreasing order, each combustion score in the ordered list corresponding to a material and positioned at an index in the ordered list, wherein the index represents a suitability rank for the material to be used as a fuel in a combustion engine.

9. The method of claim 8, wherein:
each material within the plurality of materials comprises one or more substances, wherein each substance has a concentration within the material;
the computational model comprises:
an artificial intelligence (AI) model configured to receive a representation of a substance and output a set of values of the one or more physical properties for the substance; and
a chemical model, configured to receive values for the one or more physical properties for each of the one or more substances comprised in a material and output values for the one or more physical properties for the material; and
determining the values for the one or more physical properties for a material comprises:
determining, with the AI model, values for the one or more physical properties for the one or more substances; and
determining, with the chemical model, the values for the one or more physical properties for the material from the values for the one or more physical properties for the one or more substances and the concentration of each substance within the material.

10. The method of claim 9, wherein:
the representation is a simplified molecular-input line-entry system (SMILES) representation, and
the AI model comprises:
a molecular descriptor generator configured to receive a simplified molecular-input line-entry system (SMILES) representation of a molecule and output a molecular descriptor for the molecule;
a pre-processor configured to receive the molecular descriptor from the molecular descriptor generator and output a pre-processed molecular descriptor; and
a super learner model configured to receive the pre-processed molecular descriptor from the pre-processor and output a set of values of the physical properties for the molecule, wherein the super learner model comprises one or more machine-learned models.

11. The method of claim 8, wherein the combustion engine is an ultra-lean burn engine.

12. The method of claim 8, wherein the scoring function is based on one or more factors selected from the group consisting of an efficiency factor, an emissions factor, and a performance factor.

13. A method, comprising:
obtaining a set of one or more physical properties influencing a combustion quality of a material in a combustion engine;
obtaining a vector of N substances, wherein N is an integer greater than or equal to two;
obtaining a computational model configured to receive a material composed of the N substances, and output values for the one or more physical properties for the material, wherein each substance has a concentration within the material;
obtaining a first scoring function configured to receive the values of the one or more physical properties for a material and output a combustion score for the material;
defining a first Merit function that receives a vector of N concentrations as input and returns, as output, the combustion score of a material composed of the N substances, the substance at an index of the vector of N substances having the concentration at a same index from the vector of N concentrations, wherein the combustion score is the output of the first scoring function that receives, as input, the one or more physical properties output by the computational model that receives the material as input; and
computing, with an optimizer, a first optimal vector of N concentrations, wherein the optimizer is configured to seek to maximize the first Merit function.

14. The method of claim 13, further comprising:
obtaining a second scoring function configured to receive the values of the one or more physical properties for a material and output a combustion score for the material;
defining a second Merit function that receives a vector of N concentrations as input and returns, as output, the combustion score of a material composed of the N substances, the substance at an index of the vector of N substances having the concentration at the same index from the vector of N concentrations, wherein the combustion score is the output of the second scoring function that receives, as input, the one or more physical properties output by the computational model that receives the material as input;
computing, with the optimizer, a second optimal vector of N concentrations, wherein the optimizer is configured to seek to maximize the second Merit function; and
defining a vector of N concentration ranges, wherein a minimum of the concentration range at each index of the vector of N concentration ranges is the minimum between the value of the first optimal vector of N concentrations at the same index and the value of the second optimal vector of N concentrations at the same index, and a maximum of the concentration range at each index of the vector of N concentration ranges is the maximum between the value of the first optimal vector of N concentrations at the same index and the value of the second optimal vector of N concentrations at the same index.

15. The method of claim 13, wherein obtaining the computational model comprises:
determining an artificial intelligence (AI) model configured to receive a representation of each substance within the material and output a set of values of the one or more physical properties for each substance; and
obtaining a chemical model, configured to receive values for the one or more physical properties for each substance comprised in a material and a proportion of each substance within the material, and output values for the one or more physical properties for the material.

16. The method of claim 15, wherein:
the representation is a simplified molecular-input line-entry system (SMILES) representation,
the artificial intelligence model comprises:
a molecular descriptor generator configured to receive a simplified molecular-input line-entry system (SMILES) representation of a substance and output a molecular descriptor for the substance;
a pre-processor configured to receive the molecular descriptor from the molecular descriptor generator and output a pre-processed molecular descriptor; and
a super learner model configured to receive the pre-processed molecular descriptor from the pre-processor and output a set of values of the physical properties for the substance, wherein the super learner model comprises one or more machine-learned models, and
determining the artificial intelligence model comprises:
obtaining a plurality of training examples from a training database wherein each training example comprises:
a simplified molecular-input line-entry system (SMILES) description of a substance, and
values of one or more physical properties;
processing the plurality of training examples, with a molecular descriptor generator to produce a plurality of molecular descriptors;
obtaining a pre-processed plurality of molecular descriptors by pre-processing, with the pre-processor, the plurality of molecular descriptors;
training one or more machine-learned models using the pre-processed plurality of molecular descriptors and the training database, wherein each of the one or more machine-learned models are configured to accept a pre-processed molecular descriptor and return predictions for the values of the one or more physical properties;
scoring the one or more machine-learned models, wherein upon scoring each of the one or more machine-learned models has a score;
selecting a subset of the one or more machine-learned models, wherein each of the machine-learned models in the subset has a better score than the machine-learned models outside of the subset;
tuning hyperparameters of each of the machine-learned models in the subset;
determining a weight for each machine-learned model in the subset; and
forming the super learner model as a weighted average of each machine-learned model in the subset, wherein each machine-learned model in the subset is weighted in the weighted average according to its weight.

17. The method of claim 13, wherein obtaining the set of one or more physical properties comprises:
initializing the set of one or more physical properties as empty;
obtaining, for each material within a plurality of materials:
values, for the material, of one or more master properties within a set of one or more master properties; and
values, for the material, of one or more combustion properties in a combustion engine;
determining a statistic between each master property and each combustion property;
obtaining a statistic threshold; and
upon determining that the statistic between a master property and a combustion property is greater than the statistic threshold, adding the master property to the set of one or more physical properties.

18. The method of claim 13, wherein the engine is an ultra-lean burn combustion engine.

19. The method of claim 13, wherein the first scoring function is based on one or more factors selected from the group consisting of an efficiency factor, an emissions factor, and a performance factor.

20. The method of claim 15, wherein the value of each physical property for the material, within the one or more physical properties, output by the chemical model, is a weighted average of the values for the physical property for the one or more substances, wherein the weight for the value for the physical property of each substance is the concentration of the substance within the material.
